(19) United States
(12) Patent Application Publication
(10) Pub. No.: US 2025/0331881 A1
(43) Pub. Date: Oct. 30, 2025

(54) INTRACARDIC TISSUE ENGAGEMENT SENSOR USING LIGHT OR ULTRASOUND

(71) Applicant: KONINKLIJKE PHILIPS N.V., EINDHOVEN (NL)

(72) Inventors: Thomas John McPEAK, CALEDONIA, MN (US); James JENSEN, MAPLE GROVE, MN (US); Alyssa TORJESEN, EXETER, NH (US); Jacob Paul MERTENS, MAPLE GROVE, MN (US)

(21) Appl. No.: 19/192,430

(22) Filed: Apr. 29, 2025

Related U.S. Application Data
(60) Provisional application No. 63/640,177, filed on Apr. 29, 2024.

(51) Int. Cl. A61B 17/295; A61B 17/00; A61B 17/29

(52) U.S. Cl. CPC A61B 17/295; A61B 2017/00066; A61B 2017/00106; A61B 2017/00243; A61B 2017/00292; A61B 2017/2947

(57) ABSTRACT

An apparatus includes an intracardiac tissue engagement sensor configured to sense engagement with a valve leaflet of a heart valve. The intracardiac tissue engagement sensor includes a first jaw and a second jaw configured for movement relative to one another, where the first jaw and the second jaw are configured to receive a valve leaflet in a space between the first jaw and the second jaw. The intracardiac tissue engagement sensor also includes an emitter coupled to the first jaw, where the emitter is configured to emit energy in an unmodified state, and a receiver coupled to the second jaw. The receiver is configured to receive the energy in a modified state, or not receive the energy, based on interaction with the valve leaflet that is positioned between the emitter and the receiver.

### TECHNICAL FIELD

The subject matter described herein relates to a tissue gripping device that incorporates a tissue engagement sensing capability using light and/or ultrasound.

### BACKGROUND

There are a number of structural heart procedures where it can be important to know how much tissue is enveloped by a grasping structure, such as trans-catheter edge to edge repair of a valve (TEER), or in a leaflet resection procedure in preparation for a valvular implant. Currently, physicians have to rely solely on imaging methods to gauge the amount of tissue engaged by grasping structures such as TEER devices. However, the long-term efficacy of implantable TEER devices relies heavily on sufficient tissue engagement to prevent dislodgement or complete embolization. Similarly, resection of heart valve leaflets may rely on firmly grasping the entire leaflet, which may be difficult to judge using imaging alone. X-ray (e.g., fluoroscopic) may clearly show grasping structures, but may not show the soft tissue they are intended to grasp. Soft tissue may show up well in ultrasound images that may not clearly resolve the fine details and positioning of hard structures such as intraluminal tissue gripping devices.

The information included in this Introduction section of the specification, including any references cited herein and any description or discussion thereof, is included for context and/or technical reference purposes only and is not to be regarded as subject matter by which the scope of the disclosure is to be bound or otherwise limited in any manner.

### SUMMARY

Disclosed herein is a tissue gripping device with tissue engagement sensor using light and/or ultrasound, with associated systems and methods. The device includes a flexible elongate member to introduce the device into a body lumen of a patient. The device also includes a tissue gripping capability (e.g., gripping of heart valve leaflets or other tissue), as well as a tissue engagement sensing capability that allows a clinician to determine how much tissue is engaged by the gripping device. The tissue gripping device with tissue engagement sensor has particular but not exclusive utility for intracardiac procedures such as intracardiac heart valve replacements. The present disclosure describes a method for determining the amount of tissue in between two or more jaws of a grasping device using light transmitted by one jaw that is then received by the other jaw(s). Although it may be best suited for a temporary grasping of the tissue, also described is a method to make the grasping structure implantable, and therefore, detachable from the rest of the device.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect includes an apparatus that includes an intracardiac tissue engagement sensor configured to sense engagement with a valve leaflet of a heart valve, where the intracardiac tissue engagement sensor may include: a first jaw and a second jaw configured for movement relative to one another, where the first jaw and the second jaw are configured to receive a valve leaflet in a space between the first jaw and the second jaw; a first emitter coupled to the first jaw, where the first emitter is configured to emit first energy in an unmodified state; and a first receiver coupled to the second jaw, where the first receiver is configured to receive the first energy in a modified state or not receive the first energy based on interaction with the valve leaflet that is positioned between the first emitter and the first receiver. Other examples of this aspect include corresponding computer systems, apparatus, and computer programs recorded on one or more computer storage devices, each configured to perform the actions of the methods.

Implementations may include one or more of the following features. In some aspects, when the first jaw and the second jaw are in a closed state, the first emitter and the first receiver are aligned along a length of the intracardiac tissue engagement sensor. In some aspects, the intracardiac tissue engagement sensor further may include: a second emitter disposed on the first jaw and spaced from the first emitter along a length of the first jaw; and a second receiver disposed on the second jaw and spaced from the first emitter along a length of the second jaw, where, when the first jaw and the second jaw are in the closed state, the second emitter and the second receiver are aligned along the length of the first jaw and the length of the second jaw. In some aspects, the second emitter is configured to emit second energy in the unmodified state, where the second receiver is configured to receive the second energy in the unmodified state when the valve leaflet is not positioned between the second emitter and the second receiver. In some aspects, the processor is configured to determine an amount of the engagement between the valve leaflet and the intracardiac tissue engagement sensor, based on first receiver receiving the first energy in the modified state or not receiving the first energy. In some aspects, the first energy may include at least one of light or ultrasound. In some aspects, the modified state may include a change in intensity, wavelength, or polarization as compared with the unmodified state. In some aspects, the intracardiac tissue engagement sensor is permanently coupled to the catheter. In some aspects, the intracardiac tissue engagement sensor is implantable within a heart, where the intracardiac tissue engagement sensor is removably coupled to the catheter. In some aspects, the apparatus may include a tissue cutter configured to slit the heart valve leaflet. In some aspects, the tissue cutter is controllable to cut the valve leaflet based on whether the valve leaflet is positioned between the first emitter and the first receiver. Implementations of the described techniques may include hardware, a method or process, or computer software on a computer-accessible medium.

One general aspect includes a tissue gripping and measurement device, which includes a catheter configured to be positioned within a body of a patient. The device also includes a gripping assembly coupled to the catheter and may include: a first jaw, a second jaw, a hinge mechanism coupled to the first jaw and the second jaw and configured to rotate the first jaw relative to the second jaw between an open state and a closed state. The device also includes a first emitter disposed on the first jaw or the second jaw. The device also includes a first receiver disposed on the other of the first jaw or the second jaw. When tissue is not fully gripped by the gripping assembly, first energy emitted by the first emitter is received by the first receiver in an unmodified state. When tissue is fully gripped by the gripping assembly, the first energy emitted by the first emitter is received by the first receiver in a modified state. Other examples of this aspect include corresponding computer systems, apparatus, and computer programs recorded on one or more computer storage devices, each configured to perform the actions of the methods.

Implementations may include one or more of the following features. In some aspects, the tissue gripping and measurement device may include: a second emitter disposed on the first jaw or the second jaw; and a second receiver disposed on the other of the first jaw or the second jaw, where when tissue is not gripped by the gripping assembly, second energy emitted by the second emitter is received by the second receiver in the unmodified state, and where when tissue is partially gripped by the gripping assembly, the second energy emitted by the first emitter is received by the first receiver in the modified state. In some aspects, the second energy is emitted by the second emitter at a wavelength or polarization different from the wavelength or polarization of the first energy emitted by the first emitter. In some aspects, the first emitter is an optical fiber coupled to a light source, and the first receiver is an optical fiber coupled to a light sensor. In some aspects, the first emitter is an LED, and the first receiver is a photodiode. In some aspects, the first emitter is an ultrasound emitter, and the first receiver is an ultrasound receiver. In some aspects, the modified state may include a change in intensity, wavelength, or polarization as compared with the unmodified state. In some aspects, the tissue is a heart valve leaflet, and where the gripping assembly is configured to grip the heart valve leaflet. In some aspects, the gripping assembly, the first emitter, and the first receiver are configured to remain within the body after gripping the heart valve leaflet. In some aspects, the hinge mechanism may include a hinge pin and a pull wire. In some aspects, the processor is configured to detect whether the tissue is fully gripped by the gripping assembly based on whether the first energy received by the first receiver is received in the modified state or the unmodified state. In some aspects, the tissue gripping and measurement device may include a tissue cutter configured to cut the tissue. In some aspects, the tissue cutter is controllable to cut the tissue based on whether the first energy emitted by the first emitter is received by the first receiver in a modified state. Implementations of the described techniques may include hardware, a method or process, or computer software on a computer-accessible medium.

This Summary is provided to introduce a selection of concepts in a simplified form that are further described below in the Detailed Description. This Summary is not intended to identify key features or essential features of the claimed subject matter, nor is it intended to limit the scope of the claimed subject matter. A more extensive presentation of features, details, utilities, and advantages of aspects of the present disclosure, e.g., as defined in the claims, is provided in the following written description of various examples and/or aspects of the disclosure and illustrated in the accompanying drawings.

### BRIEF DESCRIPTION OF THE DRAWINGS

Illustrative aspects of the present disclosure will be described with reference to the accompanying drawings, of which:

FIG. 1A is a side view of a human heart, according to aspects of the present disclosure.

FIG. 1B is a cross-sectional side view of a human heart, according to aspects of the present disclosure.

FIG. 2A is a cross-sectional side view of a human heart undergoing a mitral valve transcatheter edge-to-edge repair (TEER) procedure, according to aspects of the present disclosure.

FIG. 2B is a close-up view of the TEER procedure of FIG. 2A, according to aspects of the present disclosure.

FIG. 3 is a schematic, diagrammatic representation, in block diagram form, of an example system, according to aspects of the present disclosure.

FIG. 4A is a schematic, diagrammatic side view of an example intraluminal tissue engagement sensor device in a closed configuration, according to aspects of the present disclosure.

FIG. 4B is a schematic, diagrammatic side view of an example intraluminal tissue engagement sensor device in an open configuration, according to aspects of the present disclosure.

FIG. 5 is a schematic, diagrammatic, end cross-sectional view of an example intraluminal tissue engagement sensor device, taken along cut line 5-5 of FIG. 4A, according to aspects of the present disclosure.

FIG. 6 is a schematic, diagrammatic side view of an example intraluminal tissue engagement sensor device partially gripping tissue, according to aspects of the present disclosure.

FIG. 7 is a schematic, diagrammatic side view of an example intraluminal tissue engagement sensor device fully gripping tissue, according to aspects of the present disclosure.

FIG. 8 is a schematic, diagrammatic representation, in block diagram form, of an example tissue engagement measurement process, according to aspects of the present disclosure.

FIG. 9 is a schematic, diagrammatic representation, in block diagram form, of an example tissue engagement measurement process, according to aspects of the present disclosure.

FIG. 10 is a schematic, diagrammatic representation, in block diagram form, of an example tissue engagement measurement process, according to aspects of the present disclosure.

FIG. 11 is a schematic, diagrammatic view of a system according to aspects of the present disclosure.

FIG. 12 is a schematic, diagrammatic side view of an example intraluminal tissue engagement sensor device, according to aspects of the present disclosure.

FIG. 13 is a schematic, diagrammatic side view of an example intraluminal tissue engagement sensor device, according to aspects of the present disclosure.

FIG. 14 is a schematic, diagrammatic representation, in block diagram form, of a system according to aspects of the present disclosure.

FIG. 15 is a schematic, diagrammatic side view of an example implantable heart prosthesis delivery catheter, according to aspects of the present disclosure.

FIG. 16 is a schematic, diagrammatic side view of an example implantable heart prosthesis delivery catheter partially gripping a pair of heart valve leaflets, according to aspects of the present disclosure.

FIG. 17 is a schematic, diagrammatic side view of an example implantable heart prosthesis delivery catheter fully gripping a pair of heart valve leaflets, according to aspects of the present disclosure.

FIG. 18 is a schematic, diagrammatic side view of an example implantable heart prosthesis delivery catheter separating from the implantable heart prosthesis, according to aspects of the present disclosure.

FIG. 19 is a schematic, diagrammatic representation, in block diagram form, of an example system, according to aspects of the present disclosure.

FIG. 20 is a schematic, diagrammatic side view of an example intraluminal tissue engagement sensor device, according to aspects of the present disclosure.

FIG. 21 is a schematic, diagrammatic representation, in block diagram form, of an example system, according to aspects of the present disclosure.

FIG. 22 is a schematic, diagrammatic side view of at least a portion of an example intraluminal tissue engagement sensor device, according to aspects of the present disclosure.

FIG. 23 is a schematic, diagrammatic, side cross-sectional view of an example movable tissue engagement sensor assembly, according to aspects of the present disclosure.

FIG. 24A is a diagrammatic, side cross-sectional view of an example intraluminal tissue engagement sensor device measuring tissue, according to aspects of the present disclosure.

FIG. 24B is a diagrammatic, side cross-sectional view of an example intraluminal tissue engagement sensor device measuring tissue, according to aspects of the present disclosure.

FIG. 25 is a schematic diagram of a processor circuit, according to aspects of the present disclosure.

FIG. 26 is a schematic, diagrammatic representation, in flow diagram form, of an example tissue engagement and tissue cutting method, according to aspects of the present disclosure.

FIG. 27 is a schematic, diagrammatic representation, in block diagram form, of an example tissue engagement and tissue cutting system, according to aspects of the present disclosure.

FIG. 28 is a schematic, diagrammatic representation, in block diagram form, of an example tissue engagement and tissue cutting system, according to aspects of the present disclosure.

FIG. 29 is a schematic, diagrammatic side cross-sectional view of an example tissue grasping, tissue engagement sensing, and tissue cutting device, according to aspects of the present disclosure.

FIG. 30 is a schematic, diagrammatic side cross-sectional view of an example tissue grasping, tissue engagement sensing, and tissue cutting device, according to aspects of the present disclosure.

FIG. 31 is a schematic, diagrammatic end cross-sectional view of the example tissue grasping, tissue engagement sensing, and tissue cutting device of FIG. 30, according to aspects of the present disclosure.

### DETAILED DESCRIPTION

Disclosed herein is a tissue gripping device with tissue engagement sensor, with associated systems and methods. During a heart valve replacement or other intracardiac procedure, gripping a heart valve leaflet from the center of the valve towards the annulus of the valve (or vice versa) can be advantageous for several reasons, including resection of the leaflet. A transcatheter edge to edge repair (TEER) procedure, used to treat mitral valve regurgitation, is a procedure in which the leaflets of the mitral valve are “grabbed” by a clip like device, and then drawn together as the device is deployed.

This tissue gripping device with tissue engagement sensor enables a clinician to immobilize the leaflet and then measure the amount of tissue engagement, to determine whether the gripping device is properly placed or needs to be opened, repositioned, and closed again.

The tissue gripping device with tissue engagement sensor may for example include a gripper device that is delivered endovascularly into the heart, and is configured to clamp onto a valve leaflet of interest. In an example, a flexible elongate member (e.g., a catheter) is equipped with a central lumen, a pull wire, and a set of jaws at the distal end, similar to an alligator clip or gripper type catheter. In use, the catheter is introduced into the vasculature of the patient and directed to the tissue of interest (e.g., a valve leaflet). Once the device is in position, the user opens the jaws, gains control of the tissue of interest, and then closes the jaws. If the tissue engagement sensor indicates that the jaws fully enclose the tissue of interest, and the user is thus satisfied with the amount of tissue entrapped by the jaws, as well as the location being grasped, then no repositioning of the device may be necessary. Conversely, if the tissue engagement sensor indicates that the jaws are only partly enclosing the tissue of interest, then the user may open the jaws again to reposition the device.

The device includes a light source, a fiber-optic cable that connects to the device and conducts light from the source to the distal end of the device, an emitter jaw that has a plurality of light fibers distributed along the length axis of the jaw, one or more receiver jaws, also with a plurality of light fibers along their length axis, a receiving sensor, such as a photodiode, to sense the quality of the light being transmitted through the device and the tissue which it is grasping, and software that interprets and displays the information regarding the quality of the light the sensor receives.

The device described above is introduced into the body of the patient. Once the device has been directed to the area of interest, the grasping jaws are used to engage the tissue of interest. Light emitted from the light source travels through the fiber-optic bundle out to the distal end of the device, and into the individual fibers positioned along the axis of the grasping jaw. The light is transmitted from the ends of these fibers, and either impinges on the tissue being grasped, or is emitted into the open space between the jaws. A corresponding set of fibers in the opposing jaw then are able to either gather the light that is emitted into the open space, are prevented from gathering light from the fibers where tissue is blocking the transmission, or gather light that is altered in polarity, wavelength, and/or intensity by the tissue being grasped. Those signals are then transmitted back to the sensor via either a second fiber-optic bundle, or via the same fiber-optic bundle used for the emitted source light in an alternating fashion.

In the case of light that is simply blocked by the tissue, the intensity of light will vary in proportion to the number of fibers which are not blocked by tissue, and therefore the intensity of the light signal received will measure the length along the jaw axis that is blocked by tissue, indicating the amount of tissue engagement.

In the case of a polarity or wavelength shift, the sensor compares the emitted light to the light that is received back from the device. The sensor then compares the relative intensity of the original light to the (at least two) qualities of light that are received. For example, there might be two wavelengths of light received, where one wavelength is that of the light source, and the other is the wavelength of light that is filtered through the tissue that is grasped. The relative intensity of the two wavelengths is an indication of the amount of tissue being grasped, similar to the method used above.

In the case where it is difficult to differentiate between blood and leaflet tissue based solely on the intensity of the transmitted light, multiple light sources of varying wavelengths can be used. Laser light sources, with wavelengths strategically chosen near peaks in the absorption spectrum for either blood or leaflet tissue, can be coupled to optical fibers, or multiple LEDs can be used. Each measurement location along the jaws can contain a bundle of optical fibers, where each fiber is coupled to a light source of a different wavelength. The light sources within each bundle can be activated sequentially, or can be multiplexed by modulating each source at varying frequencies and activating them simultaneously. Photodiodes located opposite from each fiber bundle can collect the light after it is transmitted, and signals of different wavelengths can be separated either temporally or through frequency analysis in the case of a multiplexed signal. The optical properties of the measured sample, and thereby the volume of blood or leaflet tissue through which the light passed, can then be modeled using various methods, such as Monte Carlo modeling.

In the case where the grasping jaws are part of an implantable device (e.g., a mitral valve clip or other implantable device), a fiber-optic coupling can be incorporated at the desired junction between the implantable portion and the disposable/reusable portion of the device. Such a junction could be connected in a number of fashions, such as a threaded joint, a breakable connection, etc.

The methods described above can also be implemented using ultrasound energy, where the amount of energy transmitted between the jaws is analogous to the amount of light, and a phase change in the ultrasound signal caused by contact with tissue is analogous to a phase change in the light conducted across the gap in the jaws.

The present disclosure aids substantially in medical procedures such as intracardiac heart valve replacement, by improving a clinician's ability to determine how much tissue is engaged by a gripping device. Implemented on a catheter in association with a catheter system, the tissue gripping device with tissue engagement sensor disclosed herein provides practical, precise surgical capabilities in an intraluminal, intravascular or intracardiac environment. This improved tissue gripping technology transforms a complex, potentially imprecise procedure requiring high levels of skill and training into a repeatably precise tissue gripping technique, without the normally routine need to achieve this precision through open-chest surgical interventions. This unconventional approach improves the functioning of the intraluminal, intravascular, or intracardiac catheter system, by allowing a greater range of procedures to be reliably performed using intraluminal, intravascular, or intracardiac techniques.

Control of the tissue gripping device with tissue engagement sensor may be implemented at least partially through ultrasound or X-ray imaging under the control of a processor and viewable on a display, and operated by a control process executing on a processor that accepts user inputs from a keyboard, mouse, or touchscreen interface. In that regard, the control process performs certain specific operations in response to different inputs or selections made at different times. Structures, functions, and operations of the processor, display, sensors, and user input systems can enable novel features or aspects of the present disclosure.

For the purposes of promoting an understanding of the principles of the present disclosure, reference will now be made to the examples illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one example and/or aspect may be combined with the features, components, and/or steps described with respect to other examples and/or aspects of the present disclosure. Additionally, while the description below may refer to blood vessels, it will be understood that the present disclosure is not limited to such applications. For example, the devices, systems, and methods described herein may be used in any body chamber or body lumen, including an esophagus, veins, arteries, intestines, ventricles, atria, or any other body lumen and/or chamber. For the sake of brevity, however, the numerous iterations of these combinations will not be described separately.

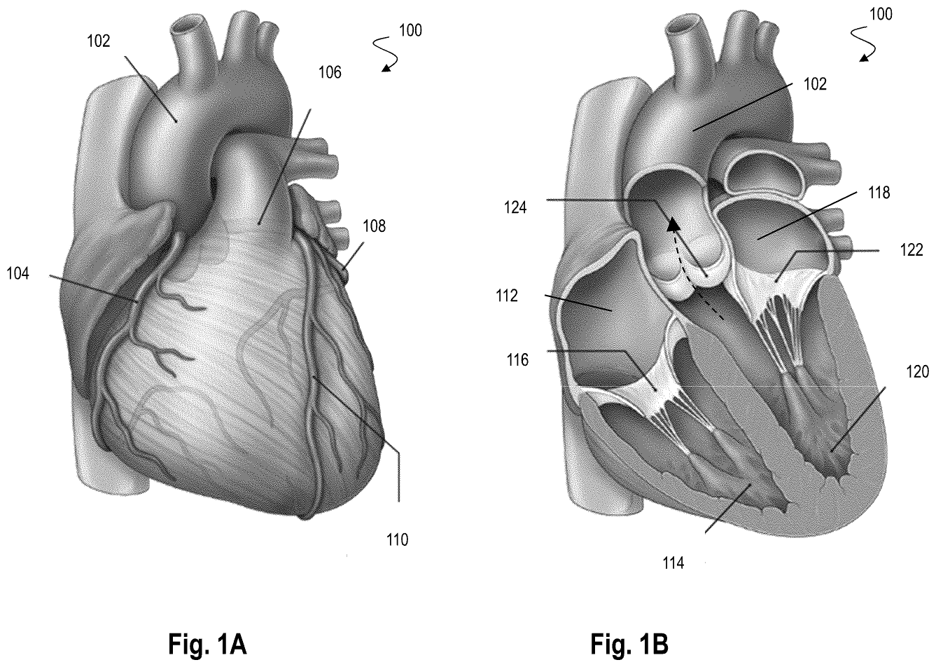

FIG. 1A is a side view of a human heart 100 according to aspects of the present disclosure. Visible are an aorta 102 from which stems a right coronary artery 104 and a left main coronary artery 106. The left main coronary artery 106 branches into a left circumflex coronary artery 108 and a left anterior descending coronary artery 110. The right coronary artery 104, the left main coronary artery 106, the left circumflex coronary artery 108, and a left anterior descending coronary artery 110 are the arteries that provide oxygen-rich blood to muscles of the human heart 100.

FIG. 1B is a cross-sectional side view of a human heart 100 according to aspects of the present disclosure. Visible are a right atrium 112 and a right ventricle 114. In that regard, oxygen-poor blood enters the human heart 100 in the right atrium 112 and travels to the right ventricle 114 through the tricuspid valve 116. The oxygen-poor blood leaves the right ventricle 114 and travels to the lungs. Also visible are a left atrium 118 and a left ventricle 120. In that regard, oxygen-rich blood is received from the lungs in the left atrium 118 and travels to the left ventricle 120 through the mitral valve 122. The oxygen-rich blood leaves the left ventricle 120 and goes out to the body through the aorta 102 via an aortic valve 124.

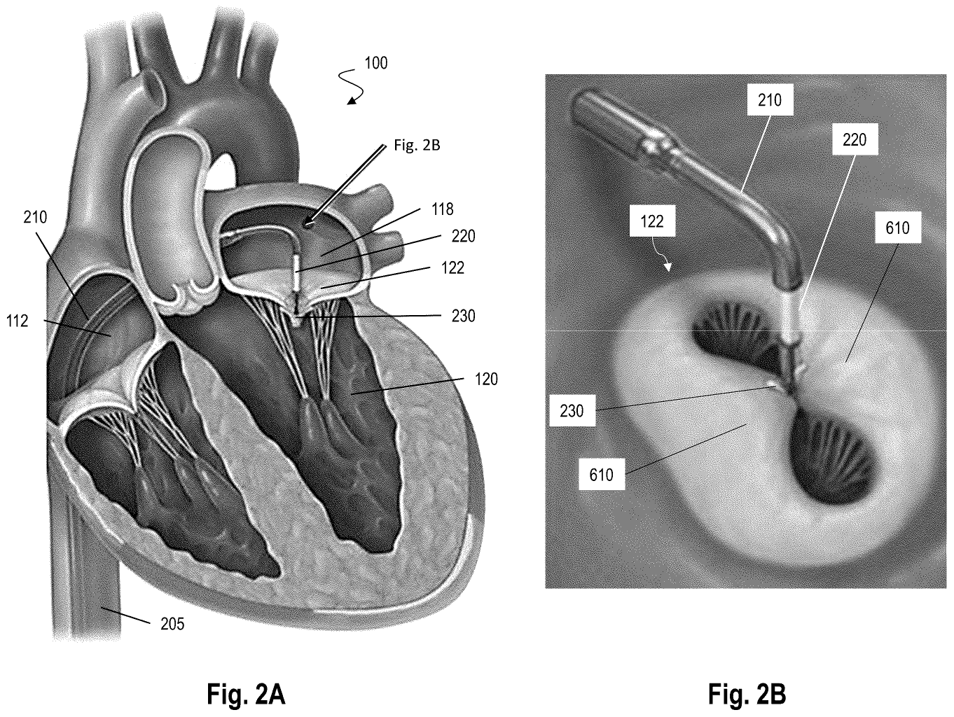

FIG. 2A is a cross-sectional side view of a human heart 100 undergoing a mitral valve transcatheter edge-to-edge repair (TEER) procedure, according to aspects of the present disclosure. Visible are the left atrium 118, left ventricle 120, and mitral valve 122. A deployment catheter 210 has entered the heart 100 through the inferior vena cava 205, through the right atrium 112, across the interatrial septum (transeptal access), and into the left atrium 118. A deployment device 220 has emerged from the catheter 210 to deploy a mitral valve clip 230, which holds together leaflets of the mitral valve 122.

FIG. 2B is a close-up view of the TEER procedure of FIG. 2A, according to aspects of the present disclosure. Visible are the catheter 210, deployment device 220, and mitral valve clip 230. The mitral valve clip 230 holds together leaflets 610 of the mitral valve 122 to treat/reduce/prevent mitral valve regurgitation.

The mitral valve TEER procedure is shown here for exemplary purposes only; it is understood that other heart valves and heart valve repair/replacement procedure types may benefit from leaflet gripping and thus fall within the scope of the present disclosure. The technology described herein may be applied to any heart prosthesis (e.g., repair device, replacement device), in or between any heart chambers, where it may be desirable to measure the engagement of tissue between two jaws or similar devices. Any location (e.g., aorta, inferior vena cava (IVC), superior vena cava (SVC), pulmonary arteries/veins, heart chamber, such as left atrium, right atrium, left ventricle, right ventricle, left atrial appendage, etc.) and/or tissue (e.g., valve, such as tricuspid valve, pulmonary valve, mitral valve, aortic valve, etc.) is contemplated. Furthermore, the tissue gripping device with tissue engagement sensor may be used in non-cardiac applications that involve gripping of tissue by a gripping device.

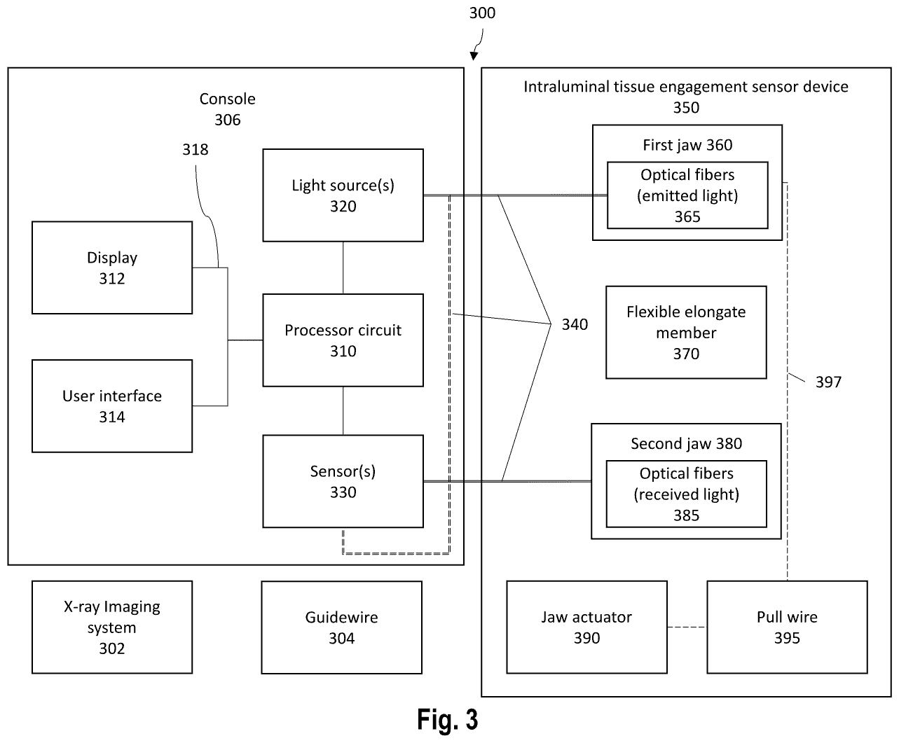

FIG. 3 is a schematic, diagrammatic representation, in block diagram form, of an example system 300 according to aspects of the present disclosure. The system 300 may be configured to evaluate (e.g., assess), display, and/or control (e.g., modify) one or more aspects of a cardiac valve immobilization. In this regard, the system 300 may be used to assess coronary vessels and/or heart tissue (e.g., the myocardium). As illustrated, the system 300 may include a processor circuit 310 in communication with a display device 312 (e.g., an electronic display or monitor), a user interface 314 (e.g., a user input device, such as a keyboard, mouse, joystick, microphone, touchscreen, and/or other controller or input device), and an intraluminal tissue engagement sensor device 350. In an example, a graphical user interface (GUI) on the display 312 shows wavelength/frequency, polarity, intensity etc. associated with the light source(s) 320 and/or the sensor(s) 330. In another example, the GUI displays an amount of tissue engagement (e.g., a length in millimeters, a percentage or fraction of the length of the gripping device, etc.). In an example, the user interface 314 provides a user input to control wavelength/frequency, intensity, polarity, etc. associated with light source(s) 320 and or sensor(s) 330. In another example, the user interface 314 allows a user interact with the GUI described above.

The processor circuit 310 is generally representative of any device suitable for performing the processing and analysis techniques disclosed herein. In some aspects, the processor circuit 310 is programmed to execute steps associated with the data acquisition, analysis, and/or instrument (e.g., device) control described herein. Accordingly, it is understood that any steps related to data acquisition, data processing, instrument control, and/or other processing or control aspects of the present disclosure may be implemented by the processor circuit 310 (e.g., computing device) using corresponding instructions stored on or in a non-transitory computer readable medium accessible by the computing device. In some instances, the processor circuit 310 is a console device. Further, it is understood that in some instances the processor circuit 310 includes one or a plurality of computing devices, such as computers, with one or a plurality of processor circuits. In this regard, it is particularly understood that the different processing and/or control aspects of the present disclosure may be implemented separately or within predefined groupings using a plurality of computing devices. Any divisions and/or combinations of the processing and/or control aspects described below across multiple computing devices are within the scope of the present disclosure. In some instances, the system 300 omits the processor circuit 310 and/or other components of the system 300 (e.g., input device 530). For example, the first jaw or emitting jaw can be manually activated/controlled by a user, and the outputs of the sensor may be interpreted by a user (e.g., via LEDs, audible tones, or other indication).

The system 300 also includes one or more light sources 320 and sensors 330. The light source(s) 320 may for example be laser light sources, LEDs, etc. The sensor(s) 330 may for example be photodiodes or other light sensing devices. The intraluminal tissue engagement sensor device 350 includes a first jaw or emitting jaw 360, which includes optical fibers 365 that emit light. The optical fibers 365 are connected to the light source 320 by a fiber optic cable 340. The intraluminal tissue engagement sensor device 350 also includes a second jaw or receiving jaw 380, which includes optical fibers 385 that receive the light emitted by the optical fibers 365. The optical fibers 385 are connected to the sensor(s) 330 by a fiber optic cable 340, which may be the same or a different fiber optic cable than the one that connects the light source(s) 320 to the fiber optic emitters 365. In some cases, the light source(s) 320 may also be connected directly to the sensor(s) 330 to provide a baseline signal. In other cases, a baseline signal may be determined through a calibration step, in which the emitting optical fibers 365 emit into the receiving optical fibers 385 with no tissue obstructing the receiving optical fibers 385.

The intraluminal tissue engagement sensor device 350 is introduced into the body via a flexible elongate member 370 such as a delivery catheter. The first jaw 360 is actuated (e.g., opened and closed relative to the second jaw 380) via a pull wire 395 connected to a jaw actuator 390, such as a knob, switch, lever, etc., while the second jaw 380 remains stationary with respect to the flexible elongate member 370.

Depending on the implementation, either the first jaw 360 or the second jaw 380 may be the emitting or receiving jaw, and either the first jaw 360 or the second jaw 380 may be actuated by the pull wire 395 via a mechanical connection 397. In some implementations, each jaw may have its own pull wire. In some implementations, each jaw may include both emitting and receiving elements. Such variations or combinations explicitly fall within the scope of the present disclosure.

The processor circuit 310 communicates with the display 312, user interface 314, light source(s) 320, and sensor(s) 330 via electrical connections 318, as part of a console 306. Also visible is a guidewire 304, which may be used to guide the flexible elongate member to a region of interest (e.g., a heart chamber or other body lumen or chamber). Also visible is an X-ray imaging system 302 which provides imaging of the patient's body to guide the movement and placement of the guidewire 304, flexible elongate member 370, and jaws 360, 380 into the region of interest, to guide the opening and closing of the jaws 360, 380, etc. Depending on the implementation, the X-ray imaging system 302 may for example be used with a contrast agent, to provide visualization of blood flow (e.g., an angiographic view), or without the contrast agent (e.g., a fluoroscopic view).

The optical fibers 365, 385 and/or the sensor(s) 330 may collectively be referred to as a tissue engagement sensor.

It is noted that block diagrams are provided herein for exemplary purposes; a person of ordinary skill in the art will recognize myriad variations that nonetheless fall within the scope of the present disclosure. For example, block diagrams may show a particular arrangement of components, subcomponents, modules, units, etc. It is understood that some embodiments of the systems disclosed herein may include additional components, that some components shown may be absent from some embodiments, and that the arrangement of components may be different than shown, while still performing the methods described herein.

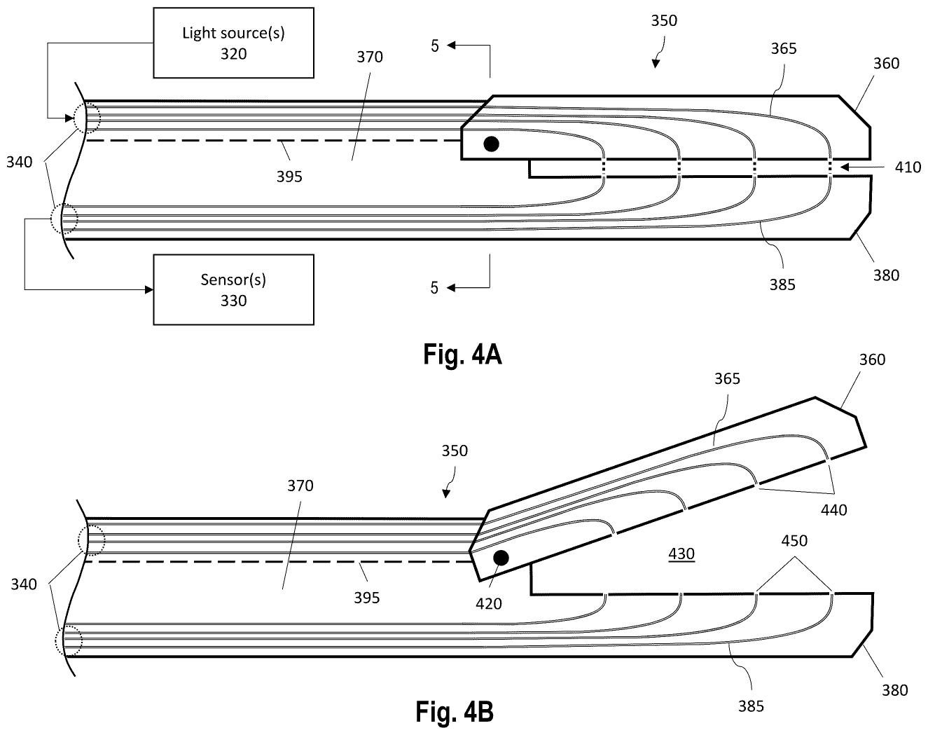

FIG. 4A is a schematic, diagrammatic side view of an example intraluminal tissue engagement sensor device 350 in a closed configuration, according to aspects of the present disclosure. Visible are the flexible elongate member 370, pull wire 395, fiber optic cables 340, first jaw, upper jaw, or emitting jaw 360, and second jaw, lower jaw, or receiving jaw 380. A fiber optic cable 340 connects the light source(s) to the emitting optical fibers 365 in the first jaw or emitting jaw 360, which emit light 410 that is received by the receiving optical fibers 385 in the second jaw or receiving jaw 380 when the first jaw or emitting jaw 360 is in its closed configuration, as shown.

FIG. 4B is a schematic, diagrammatic side view of an example intraluminal tissue engagement sensor device 350 in an open configuration, according to aspects of the present disclosure. Visible are the flexible elongate member 370, pull wire 395, fiber optic cables 340, first jaw or emitting jaw 360, and second jaw or receiving jaw 380. In the example shown in FIG. 4B, the pull wire 395 has been actuated to rotate the first jaw or emitting jaw 360 around a hinge pin 420, thus opening a gap 430 between the first jaw 360 and the second jaw 380. In this configuration, the ends or emission apertures 440 of the emitting optical fibers 365 are distant from, and not aligned with, the ends or receiving apertures 450 of the receiving optical fibers 385. Thus, light emitted by the emitting optical fibers 365 may not be received by the receiving optical fibers 385. It is noted that the number of emission apertures 440 and receiving apertures 450 may be different than shown in FIGS. 4A and 4B. In the open configuration, the jaws are prepared to receive tissue that will be grasped by the closing of the jaws. The tissue is received into the gap 430. The tissue engagement will be sensed and displayed when the jaws are in the closed configuration (e.g., as shown in FIG. 4A) with the jaws closed around tissue, when the ends or emission apertures 440 of the emitting optical fibers 365 are aligned with the ends or receiving apertures 450 of the receiving optical fibers 385 along the length of the jaws.

The first jaw 360, second jaw 380, hinge pin 420 (or other hinge mechanism), and pull wire 395 (or other actuation mechanism) may collectively be referred to as a gripping assembly. By the hinge mechanism, the first jaw and second jaw are configured to move relative to one another.

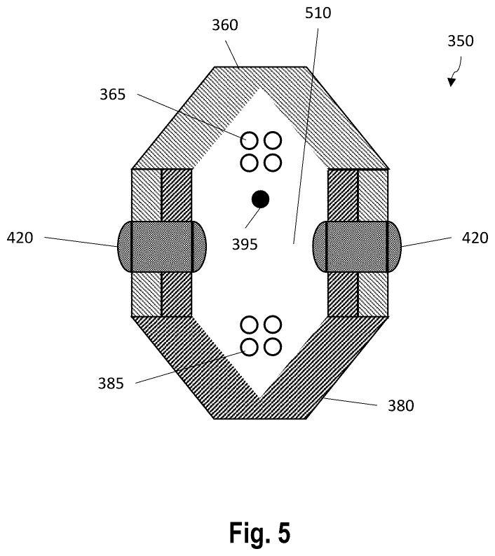

FIG. 5 is a schematic, diagrammatic, end cross-sectional view of an example intraluminal tissue engagement sensor device 350, taken along cut line 5-5 of FIG. 4A, according to aspects of the present disclosure. Visible are the upper jaw or first jaw 360, emission optical fibers 365, pull wire 395, hinge pins 420, lower jaw or second jaw 380, and receiving optical fibers 385. Although the configuration shown in FIG. 5 includes two hinge pins, a person of ordinary skill in the art will appreciate that other types of hinge may be used instead or in addition, without departing from the spirit of the present disclosure.

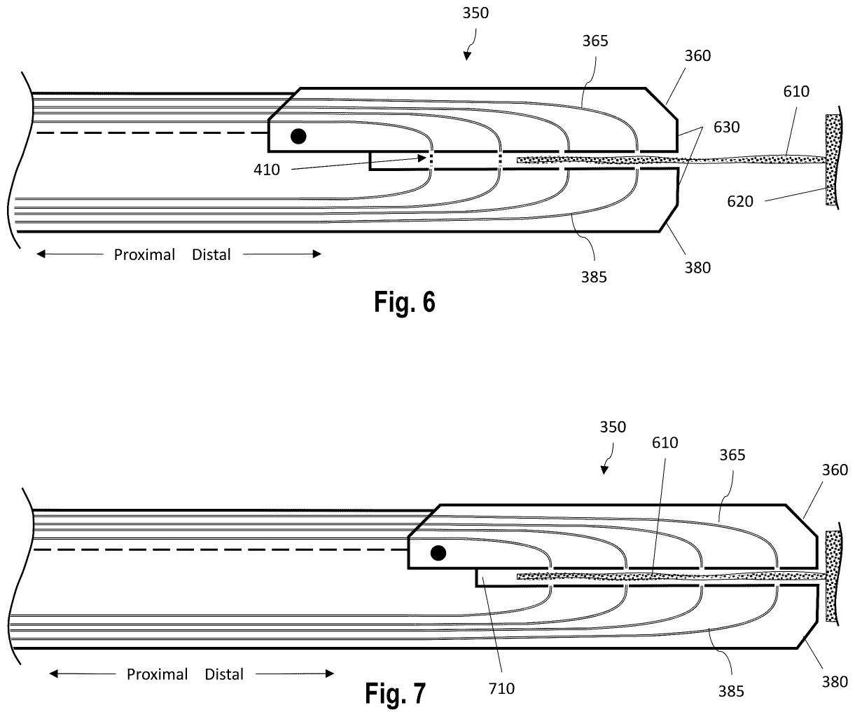

FIG. 6 is a schematic, diagrammatic side view of an example intraluminal tissue engagement sensor device 350 partially gripping tissue 610, according to aspects of the present disclosure. The tissue 610 may for example be a heart valve leaflet extending outward from the heart wall 620. In the example shown in FIG. 6, the tissue 610 is partially gripped between the first jaw 360 and second jaw 380, such that some of the emitting optical fibers 365 are blocked from emitting to their corresponding receiving optical fibers 385, such that the light is not received or is received in a modified state, whereas from other emitting optical fibers 365, light 410 is free to travel to the corresponding receiving optical fiber 385, such that the light is received in an unmodified state. Thus, for example, if each emitter-receiver pair is spaced one millimeter from its neighbors and from the distal ends 630 of the jaws, then the obstruction of two emitting optical fibers 365 would indicate that between two and three millimeters of tissue 610 are engaged by the jaws 360, 380. Known locations of the first emitters and receivers (e.g., distances from one another and/or from the proximal or distal ends of the jaws) may for example be stored in a memory of the processor circuit 310. Depending on the implementation, the user may receive an indication of partial tissue engagement as part of a screen display (e.g., displayed on the display 312), or through other methods, including but not limited to LEDs, audible tones, etc.

FIG. 7 is a schematic, diagrammatic side view of an example intraluminal tissue engagement sensor device 350 fully gripping tissue 610, according to aspects of the present disclosure. In the example shown in FIG. 7, the tissue 610 is fully gripped between the first jaw 360 and second jaw 380, such that all of the emitting optical fibers 365 are blocked from emitting to their corresponding receiving optical fibers 385. Thus, for example, if each emitter-receiver pair is spaced one millimeter from its neighbors and from the distal end and proximal end 710 of the jaws 360, 380, then the obstruction of four emitting optical fibers 365 would indicate that between four and five millimeters of tissue 610 are engaged by the jaws 360, 380. In this way, the intraluminal tissue engagement sensor device 350 can be used both to grip tissue and to determine how much tissue is being gripped. Depending on the implementation, the user may receive an indication of full tissue engagement as part of a screen display (e.g., displayed on the display 312), or through other methods, including but not limited to LEDs, audible tones, etc.

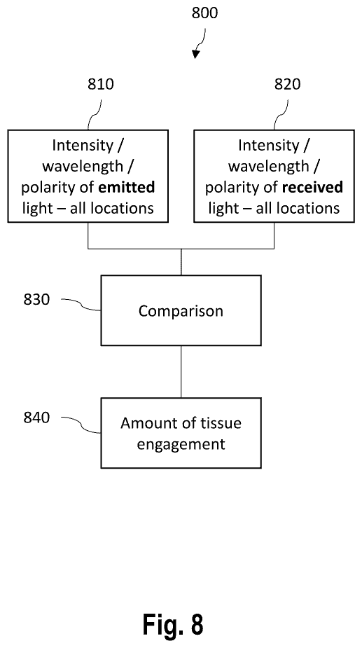

FIG. 8 is a schematic, diagrammatic representation, in block diagram form, of an example tissue engagement measurement process 800, according to aspects of the present disclosure. The process may for example be performed by the processor circuit 310. The process begins with the emitted light from all locations along the first jaw. The emitted light has an emitted intensity, wavelength, and/or polarization 810 that are known to the processor circuit, either because they are stored in memory, measured in a calibration step, or outputted directly to the sensor(s).

The emitted light is then received at all locations along the second jaw, with a received intensity, wavelength, and/or polarization 820. A comparison 830 is performed between the emitted light properties 810 and the received light properties 820. For example, if no tissue is engaged by the jaws, then the received light properties 820 should be approximately equal to the emitted light properties 810. This may occur for example in a calibration step, where the jaws are closed but no tissue is engaged. However, if opaque tissue is engaged by the jaws, then the total intensity of light received should be proportional to the amount of tissue engaged. In another example, if translucent tissue is engaged by the jaws, then the light passing through the tissue will have an altered intensity, wavelength (color), and possibly polarization. The magnitude of these differences may then be proportional to the amount of tissue engaged, and/or to the thickness or composition of the tissue.

The comparison 830 is then translated into an amount of tissue engagement 840, based on the proportionality. The amount of tissue engagement could additionally be based on the distance along length of device between ends or apertures 440, 450 (e.g., stored in memory, known to, accessible by the processor). This known distance between the ends or apertures 440, 450 can be a way to determine an absolute length (e.g., in millimeters). The amount of tissue engagement may be output by the processor circuit 310 to the display 312. The amount of tissue engagement can be the length of the tissue that is received within space between the jaws, along the length of the jaws. The amount of tissue engagement may for example be expressed as a length (e.g., in millimeters), a percentage (e.g., percent coverage of the jaws), a fraction, or any other metric that is useful to the clinician performing the medical procedure.

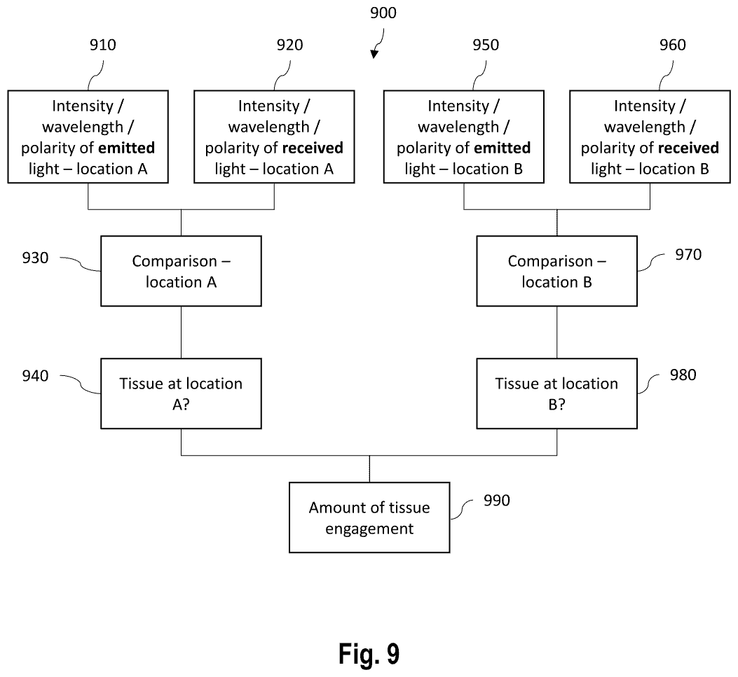

FIG. 9 is a schematic, diagrammatic representation, in block diagram form, of an example tissue engagement measurement process 900, according to aspects of the present disclosure. The process begins with the emitted light from a first location (location “A”) along the first jaw. The emitted light has an emitted intensity, wavelength, and/or polarization 910 that is stored within the memory of the processor circuit or else directly measured by the sensors. The emitted light is then received at the first location (location “A”) along the second jaw, with a received intensity, wavelength, and/or polarization 920. A comparison 890 is performed between the emitted light properties 910 and the received light properties 920 for the first location, and used in a determination step 940 to determine whether there is tissue at the first location (location “A”), e.g., because of a characteristic change in intensity, wavelength, and/or color.

Light is also emitted from a second location (location “B”) along the first jaw. The emitted light has an emitted intensity, wavelength, and/or polarization 950. The emitted light is then received at the second location (location “B”) along the second jaw, with a received intensity, wavelength, and/or polarization 960. A comparison 970 is performed between the emitted light properties 950 and the received light properties 960 for the second location, and used in a determination step 980 to determine whether there is tissue at the second location (location “B”), e.g., because of a characteristic change in intensity, wavelength, and/or color.

The determinations 940, 980 are then translated into an amount of tissue engagement 840, as described above. The amount of tissue engagement may for example be expressed as a length (e.g., in millimeters), a percentage (e.g., percent coverage of the jaws), a fraction, or otherwise.

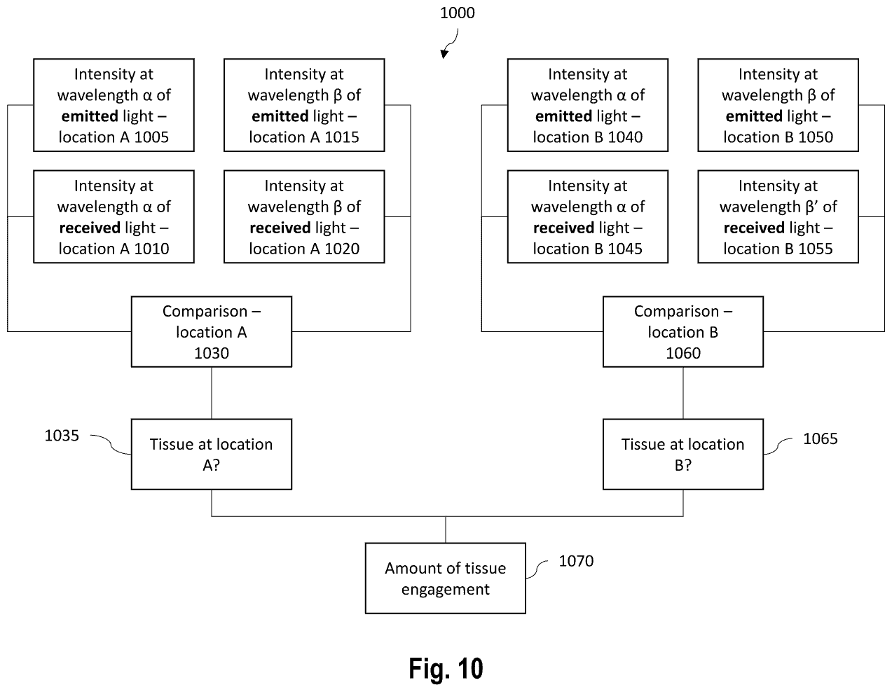

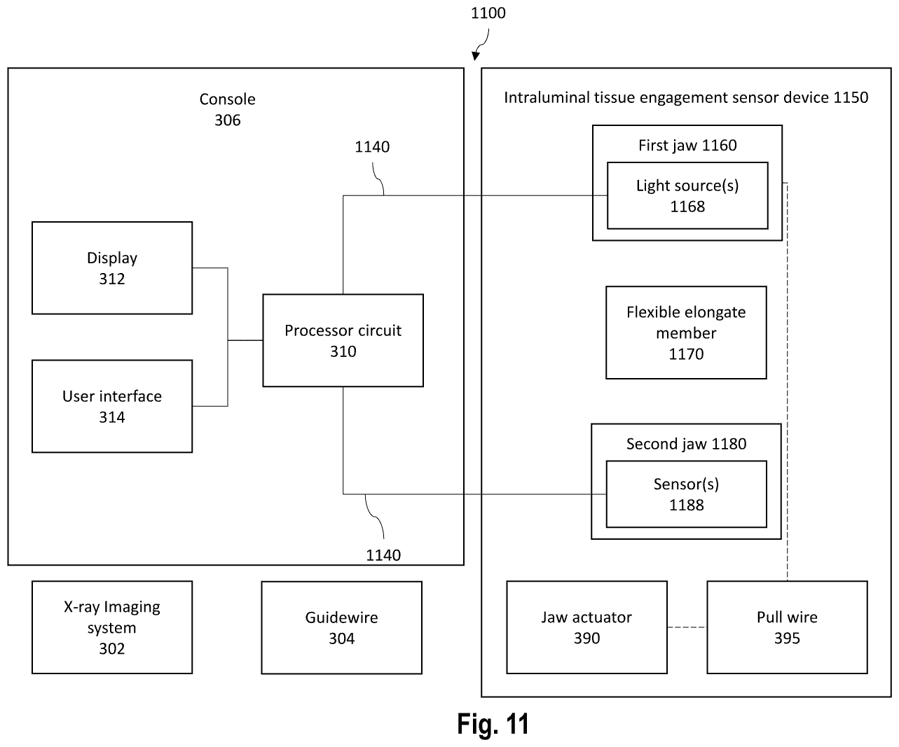

FIG. 10 is a schematic, diagrammatic representation, in block diagram form, of an example tissue engagement measurement process 1000, according to aspects of the present disclosure. The process begins with the emitted light at a first wavelength (wavelength “a”) from a first location (location “A”) along the first jaw. The emitted light at the first wavelength has an emitted intensity that is known to the processor circuit as described above. The emitted light at the first wavelength is then received at the first location (location “A”) along the second jaw, with a received intensity 1010. Light is also emitted from the first location at a second wavelength (wavelength “B”) with an intensity 1015, and received at the first location on the second jaw with a received intensity 1020.

A comparison 1030 is performed between the emitted light properties 1005, 1015 and the received light properties 1010, 1020 for the first location, and used in a determination step 940 to determine whether there is tissue at the first location (location “A”), e.g., because of a characteristic change in intensity at the first and second wavelengths. In an example light at one wavelength may pass through blood and/or tissue more readily than light at the other wavelength, resulting in a two-wavelength spectrum that can distinguish between air/water/saline (transparent), blood, tissue, and an open-jaw condition, and/or may provide information about the thickness and/or composition of the tissue.

Light is also emitted from a second location (location “B”) along the first jaw. The emitted light at the first wavelength (wavelength “a”) has an emitted intensity 1040. The emitted light is then received at the second location (location “B”) along the second jaw, with a received intensity 1045. Light is also emitted from the second location (location “B”) at the second wavelength (wavelength “B”) with an intensity 1050, and received with an intensity 1055.

A comparison 1030 is performed between the emitted light properties 1040, 1050 and the received light properties 1045, 1055 for the second location (location “B”), and used in a determination step 1060 to determine whether there is tissue at the first location (location “B”), e.g., because of a characteristic change in intensity at the first and second wavelengths.

The determinations 1035, 1065 are then translated into an amount of tissue engagement 1070, as described above. The amount of tissue engagement may for example be expressed as a length (e.g., in millimeters), a percentage (e.g., percent coverage of the jaws), a fraction, or otherwise.

In an example, the light at wavelength α may be generated by a first light source, and the light at wavelength β may be generated by a second light source. Similarly, the light at wavelength α may be detected, sensed, or measured by a first sensor or set of sensors, and the light at wavelength β may be detected, sensed, or measured by the first sensor or set of sensors, or by a second sensor or set of sensors. The two wavelengths may be carried on the same or different optical fibers.

It is noted that the process 1000 could also work using two different polarizations of light rather than two different wavelengths of light, where the tissue is known to have a measurable, repeatable effect on the polarization of light passing through it. The light source(s) 1168 and the sensor(s) 1188 may collectively be referred to as a tissue engagement sensor.

FIG. 11 is a schematic, diagrammatic representation, in block diagram form, of an example system 1100, according to aspects of the present disclosure. The system 1100 is similar to the system 300 of FIG. 3, except that the light source(s) 1168 and sensor(s) 1188 have been moved onto the intraluminal tissue engagement sensor device 350 itself. In particular, the light source or light sources 1168 are located on the first jaw, upper jaw, or emitting jaw 1160, and the sensor or sensors 1188 are located on the second jaw, lower jaw, or receiving jaw 1180. The light source(s) 1168 and sensor(s) 1188 are powered and controlled by electrical cables 1140 coupled to the processor circuit 310. Also visible are the X-ray imaging system 302, guidewire 304, console 306, processor circuit 310, display 312, user interface 314, flexible elongate member 1170, jaw actuator 390, and pull wire 395. The light sources 1168 may for example be LEDs, and the light sensors 1188 may for example be photodiodes.

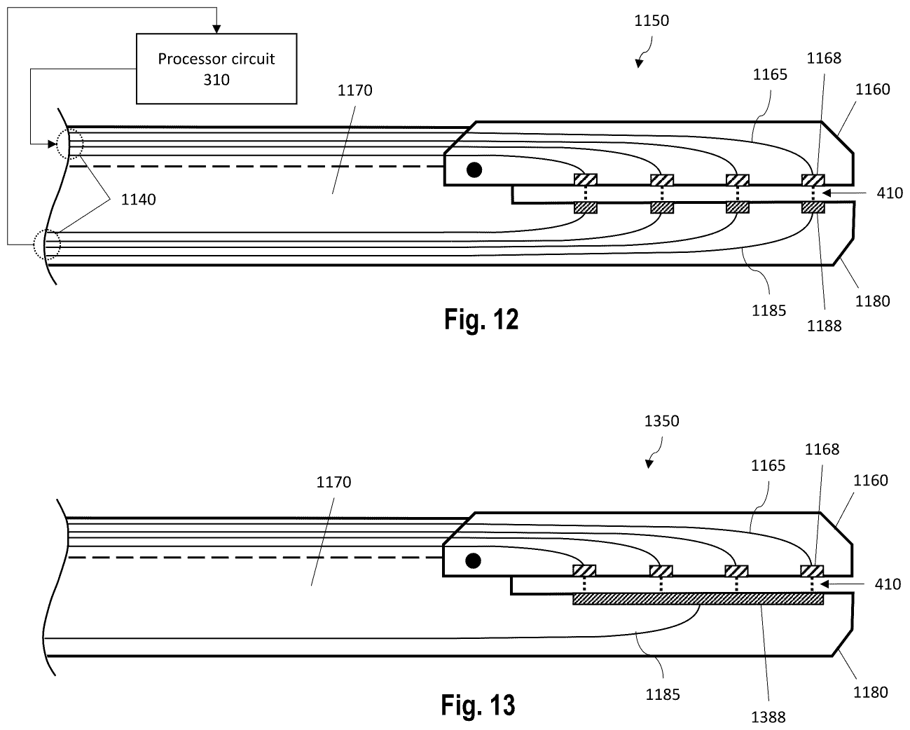

FIG. 12 is a schematic, diagrammatic side view of an example intraluminal tissue engagement sensor device 1150, according to aspects of the present disclosure. Visible are the flexible elongate member 1170, electrical cables 1140, first jaw, upper jaw, or emitting jaw 1160, and second jaw, lower jaw, or receiving jaw 1180. Electrical wires 1165 connect an electrical cable 1140 to light sources 1168 in the first jaw or emitting jaw 1160, which emit light 410. The light 410 is received by sensors 1188 in the second jaw or receiving jaw 380 when the first jaw or emitting jaw 360 is in its closed configuration, as shown. The sensors 1188 are connected to an electrical cable 1140 by electrical wires 1185 to power and control the sensors 1188. The electrical cables 1140 are connected to the processor circuit 310, which provides power and control for the light sources 1168 and light sensors 1188.

FIG. 13 is a schematic, diagrammatic side view of an example intraluminal tissue engagement sensor device 1350, according to aspects of the present disclosure. Visible are the flexible elongate member 1170, electrical cables 1140, first jaw, upper jaw, or emitting jaw 1160, and second jaw, lower jaw, or receiving jaw 1180. Electrical wires 1165 connect an electrical cable 1140 to light sources 1168 in the first jaw or emitting jaw 1160, which emit light 410. The light 410 is received by a single sensor 1388 in the second jaw or receiving jaw 380 when the first jaw or emitting jaw 360 is in its closed configuration, as shown. The sensor 1388 is connected to an electrical cable 1140 by electrical wires 1185 to power and control the sensors 1188. The electrical cables 1140 are connected to the processor circuit 310, which provides power and control for the light sources 1168 and sensor 1388. Total intensity, wavelength, or polarization of the light received by the sensor 1388 can indicate the degree of tissue engagement in a manner similar to FIG. 8.

Other configurations are possible, including a single light source and multiple light sensors, a single light source and a single light sensor, etc. In some aspects, a single emitter and a single receiver may be configured to slide on a track of known length to provide the measurements described above. In some aspects, the light emitters 1168 may be replaced with field generating electrodes, and the light sensors 1188 or 1388 may be replaced with field sensing electrodes. Such permutations explicitly fall within the scope of the present disclosure.

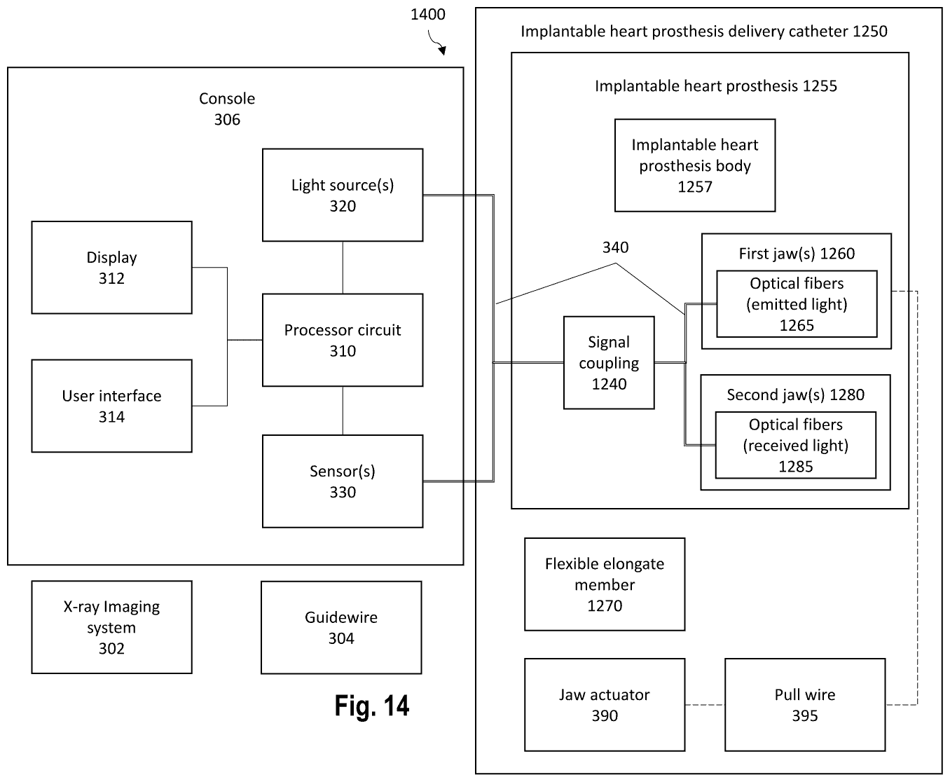

FIG. 14 is a schematic, diagrammatic representation, in block diagram form, of a system 1400 according to aspects of the present disclosure. Visible, and similar to FIG. 3, are the X-ray imaging system 302, guidewire 304, console 306, processor circuit 310, display 312, user interface 314, light source(s) 320, sensor(s) 330, and fiber optic cables 340. However, the intraluminal tissue engagement sensor device 350 has been replaced by an implantable heart prosthesis delivery catheter 1250. The implantable heart prosthesis delivery catheter 1250 includes not only a flexible elongate member 1270, jaw actuator 390, and pull wire, but also an implantable heart prosthesis 1255 that includes an implantable heart prosthesis body 1257, detachable signal coupling 1240, two first jaws 1260 with emitting optical fibers 1265, and two second jaws 1280 with receiving optical fibers 1285. The implantable heart prosthesis 1255 is designed to, for example, clamp two or more heart leaflets and then remain in the body.

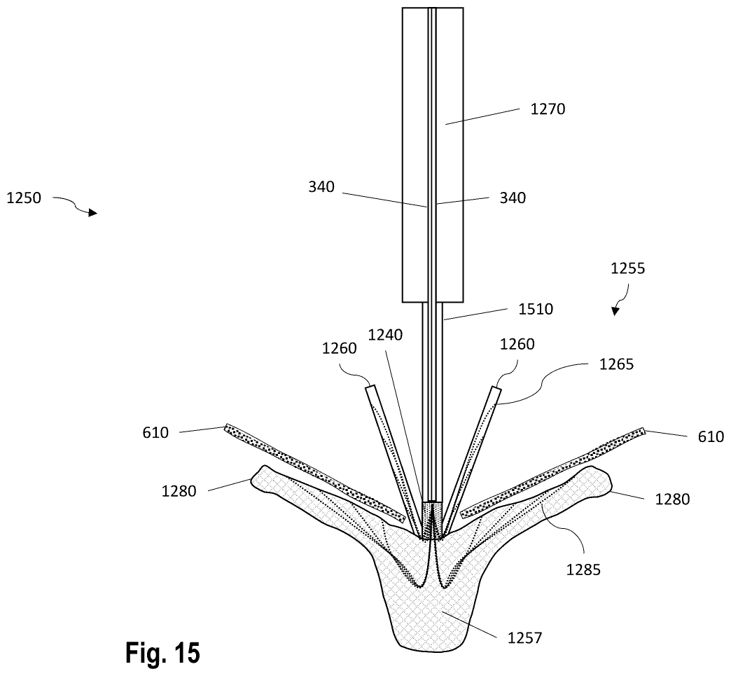

FIG. 15 is a schematic, diagrammatic side view of an example implantable heart prosthesis delivery catheter 1250, according to aspects of the present disclosure. Visible are the flexible elongate member 1270, fiber optic cables 340, a retractable deployment device 1510, and the implantable heart prosthesis 1255. The implantable heart prosthesis 1255 includes a detachable signal coupling 1240, two first jaws 1260 with emitting optical fibers 1265, two second jaws 1280 with receiving optical fibers 1285, and an implantable heart prosthesis body 1257. The emitting optical fibers 1265 and receiving optical fibers 1285 connect with the fiber optic cables 340 in the detachable signal coupling 1240. In the example shown in FIG. 15, the jaws 1260, 1280 have been positioned over a pair of heart valve leaflets 610.

The implantable heart prosthesis 1255 is introduced into the body via the flexible elongate member 1270 (e.g., a delivery catheter). The first jaws 1260 are actuated (e.g., opened and closed relative to the second jaws 1280) via a pull wire connected to a jaw actuator such as a knob, switch, lever, etc., while the second jaws 1280 remain stationary with respect to the flexible elongate member 1270. In the example shown in FIG. 15, the implantable heart prosthesis is a mitral valve clip or similar device. However, in other aspects the implantable heart prostheses may be a clip or replacement valve for any heart valve.

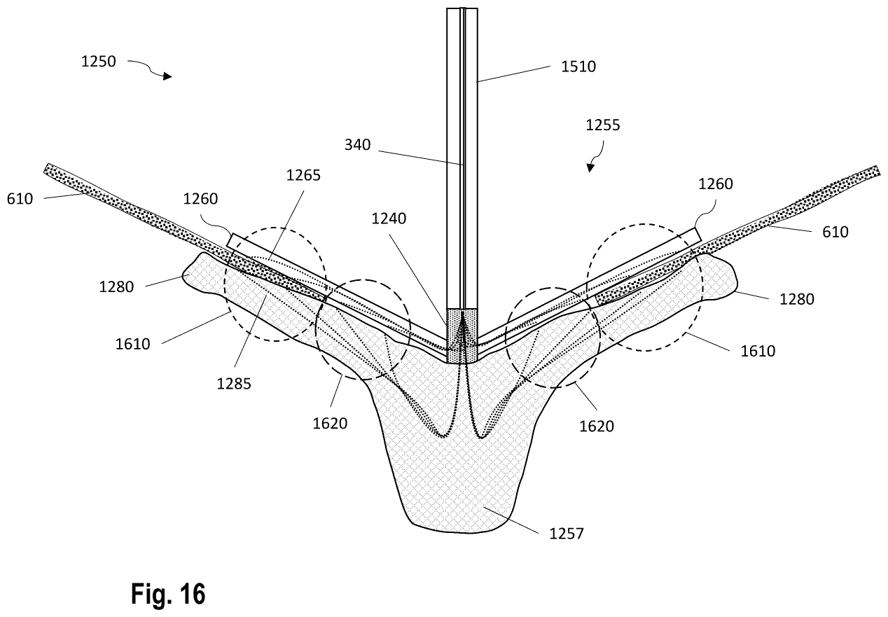

FIG. 16 is a schematic, diagrammatic side view of an example implantable heart prosthesis delivery catheter 1250 partially gripping a pair of heart valve leaflets 610, according to aspects of the present disclosure. Visible are the retractable deployment device 1510, fiber optic cables 340, implantable heart prosthesis 1255, and implantable heart prosthesis body 1257. In the example shown in FIG. 16, the upper jaws 1260 have been closed against the lower jaws 1280, partially enclosing the heart valve leaflets 610 such that in first regions 1610, the emission fiber optic cables 1265 are blocked from the receiving optical fibers 1285, while in second regions 1620, the emission fiber optic cables 1265 are not blocked from the receiving optical fibers 1285. Thus, as described above the amount of tissue engagement can be measured (e.g., in millimeters, or as a percentage of the length of the jaws), and a clinician can readily see (e.g., via a screen display on the display 312) that the hear valve leaflets 610 are not fully engaged by the jaws 1260, 1280 of the implantable heart prosthesis 1255, and therefore that it may be desirable to reopen, reposition, and re-close the jaws 1260, 1280.

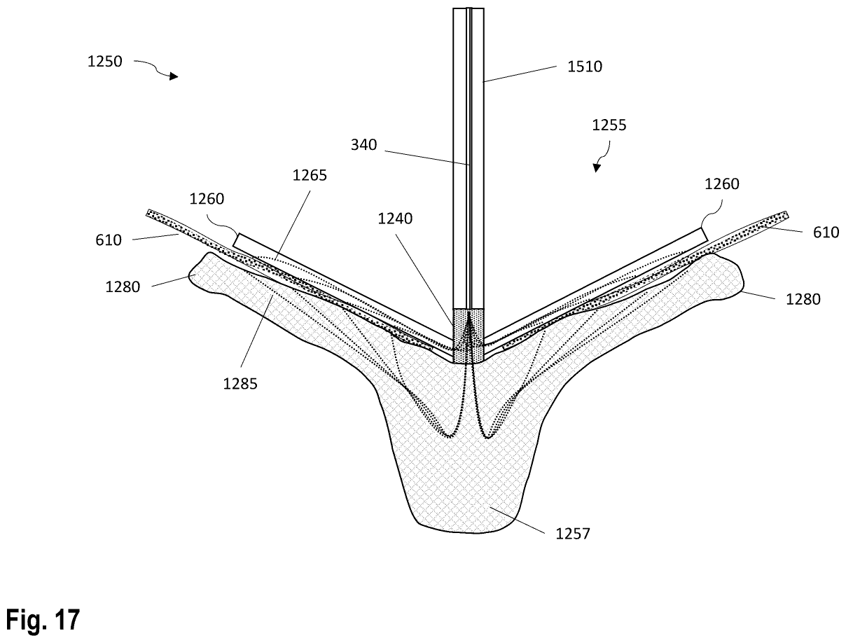

FIG. 17 is a schematic, diagrammatic side view of an example implantable heart prosthesis delivery catheter 1250 fully gripping a pair of heart valve leaflets 610, according to aspects of the present disclosure. Visible are the retractable deployment device 1510, fiber optic cables 340, implantable heart prosthesis 1255, and implantable heart prosthesis body 1257. In the example shown in FIG. 17, the upper jaws 1260 have been closed against the lower jaws 1280, fully enclosing the heart valve leaflets 610 such that all of the emitting optical fibers 1265 are covered by the leaflet tissue 610, so that their light cannot be received (or is received in altered form) by the receiving optical fibers 1285. This may represent a correct implantation of the implantable heart prosthesis, such that no repositioning is needed.

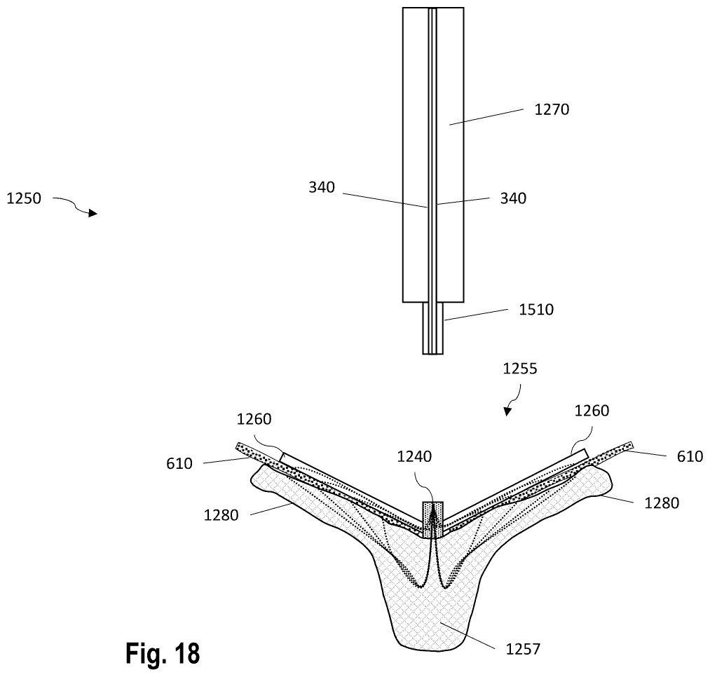

FIG. 18 is a schematic, diagrammatic side view of an example implantable heart prosthesis delivery catheter 1250 separating from the implantable heart prosthesis 1255, according to aspects of the present disclosure. The upper jaws 1260 and lower jaws 1280 are in the closed position and are fully enclosing the heart valve leaflets 610, such that the light received by the receiving optical fibers 1285 from the emitting optical fibers 1265 was fully blocked or altered by the heart valve leaflets 610. Since the implantable heart prosthesis 1255 is now correctly placed, the retractable deployment device 1510 is separated from the detachable signal coupling 1240. This separation may involve breaking a breakaway connection, unscrewing a screw connection, unlatching a latch connection, or other detachment mechanism as would occur to a person of ordinary skill in the art.

In the detached configuration, because the tissue engagement sensing capability of the implantable heart prosthesis 1255 is no longer needed, the detachable signal coupling 1240 no longer receives light from nor sends light to the fiber optic cables 340. The tissue engagement sensing capability of the implantable heart prosthesis 1255 thus no longer functions, and the implantable heart prosthesis 1255 remains behind inside the body (e.g., inside the heart) while the flexible elongate member 1270 is removed from the body.

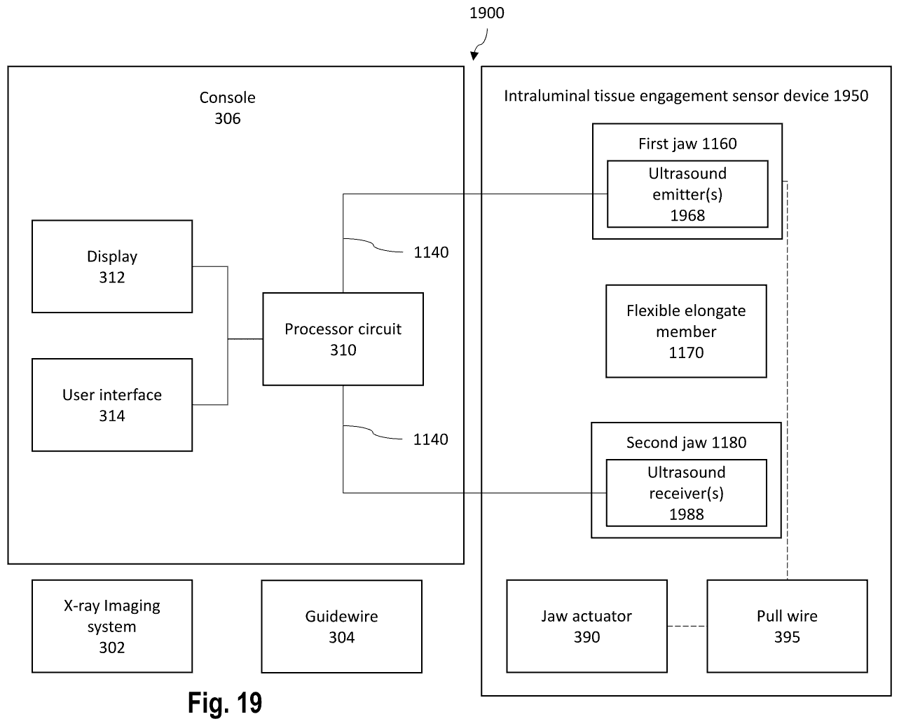

FIG. 19 is a schematic, diagrammatic representation, in block diagram form, of an example system 1900, according to aspects of the present disclosure. The system 1900 is similar to the system 1100 of FIG. 11, except that the light sources 1168 have been replaced with ultrasound emitters 1968, and the light sensors 1188 have been replaced with ultrasound receivers 1988. The ultrasound emitters 1968 may be of the same design, similar design, or different design than the ultrasound receivers 1988, and may for example be ultrasound transducers or groups of transducers incorporating lead zinc titanate (PZT), capacitive micromachined ultrasound transducer (CMUT), or piezoelectric micromachined ultrasound transducer (PMUT) transducer elements. Operation of the system 1900 may also be similar to that of the system 1100 of FIG. 11, except that tissue engagement is not measured through changes in the intensity, wavelength, or polarization of light. Rather, tissue engagement is measured through changes in the intensity or frequency of ultrasound waves, such that when the jaws 1160, 1180 are closed, if a given ultrasound receiver 1988 receives ultrasound waves at a substantially different intensity and/or frequency (e.g., greater than a threshold difference) than the intensity or frequency emitted by the corresponding ultrasound emitter 1968, then that particular ultrasound receiver 1988 may be considered to be blocked by tissue.

The ultrasound emitter(s) 1968 and the ultrasound receiver(s) 1988 may collectively be referred to as a tissue engagement sensor.

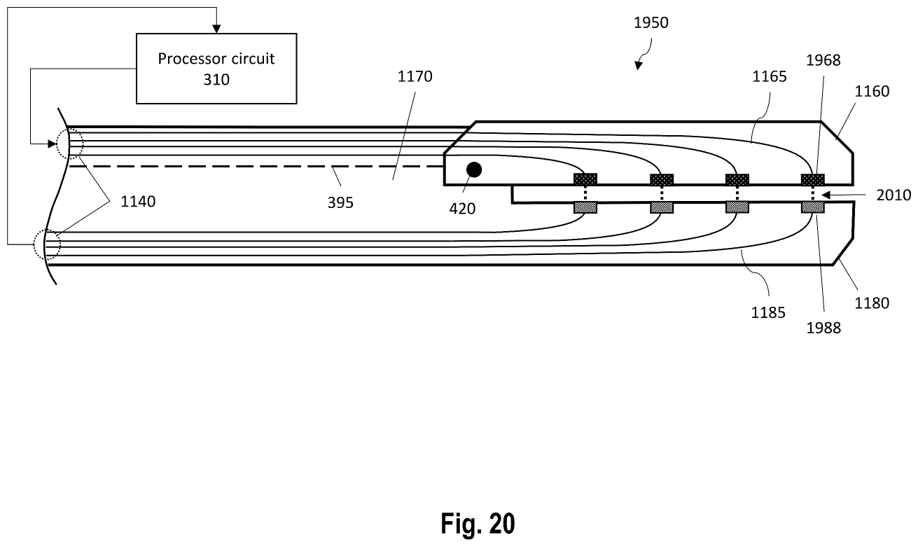

FIG. 20 is a schematic, diagrammatic side view of an example intraluminal tissue engagement sensor device 1950, according to aspects of the present disclosure. Visible are the processor circuit 310, electrical cables 1140, pull wire 395, hinge pin 420, upper jaw, emitting jaw, or first jaw 1160, transmitting electrical wires 1165, ultrasound emitters 1968, lower jaw, receiving jaw, or second jaw 1180, receiving electrical wires 1185, and ultrasound receivers 1988. Also visible are the ultrasound energy/waves 2010 transmitted between ultrasound emitters 1968 and their corresponding ultrasound receivers 1988.

More generally, the ultrasound emitters 1968 may be replaced with emitters of other types of energy (e.g., light, heat, vibration, etc.), and the ultrasound receivers may be replaced with receivers of other types of energy, provided that tissue placed in between the first emitter and receiver produces a measurable, reasonably repeatable change in at least one characteristic of the energy, such that the presence of tissue in between the first emitter and receiver can be deduced. In some aspects, more than one type of emitter and more than one type of receiver may be used. In some aspects, emitters may be on either or both jaws, and their corresponding receivers may each be positioned on the opposite jaw from the corresponding emitter.

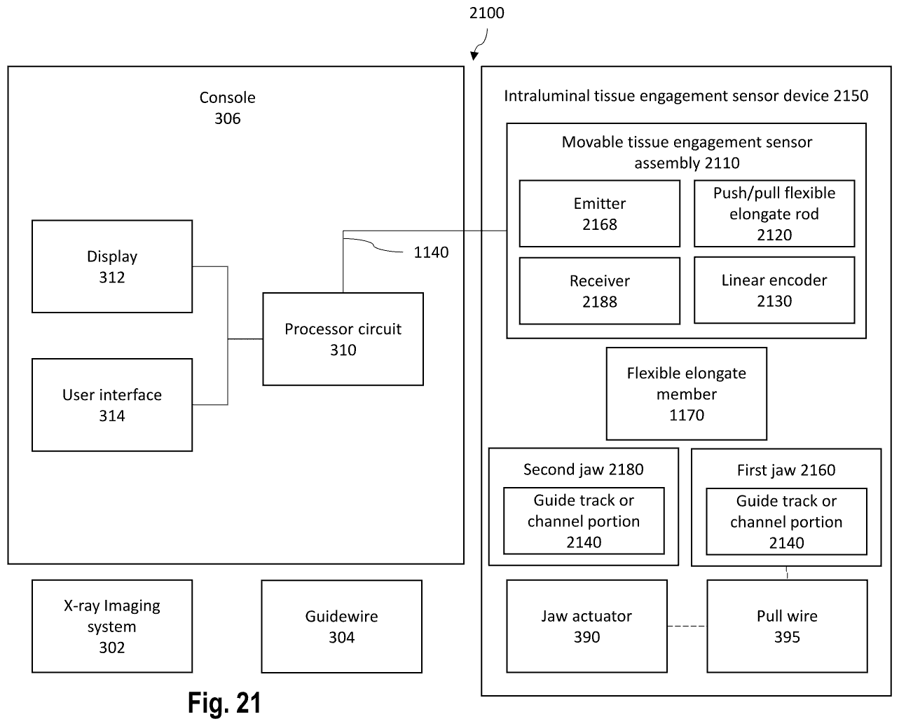

FIG. 21 is a schematic, diagrammatic representation, in block diagram form, of an example system 2100, according to aspects of the present disclosure. The system 2100 is similar to the system 1900 of FIG. 19, except that there is only a single emitter 2168, and a single receiver 2188. The system 2100 includes an intraluminal tissue engagement sensor device 2150, which includes a movable tissue engagement sensor assembly 2110. Operation of the system 2100 may also be similar to that of the system 1900 of FIG. 19, except that tissue engagement is measured by pulling back the single emitter 2168 and single receiver 2188 with a pusher/puller flexible elongate rod 2120, with the distance traveled by the emitter 2168 and receiver 2188 being measured by a linear encoder 2130. Both the first jaw 2160 and the second jaw 2180 include a guide track or channel portion 2140, through which the flexible elongate rod 2120, emitter 2168, and receiver 2188 can travel.

The emitter 2168 and receiver 2188 may use light, ultrasound, or other energy as described above. In some aspects, there may be multiple emitters and multiple receivers, or other combinations as described above. The processor circuit 310 is in communication with the linear encoder 2130 via the cable 1140 to measure the position of the push/pull rod 2120 during movement of the rod along its longitudinal axis within the guide tracks or channel portions 2140.

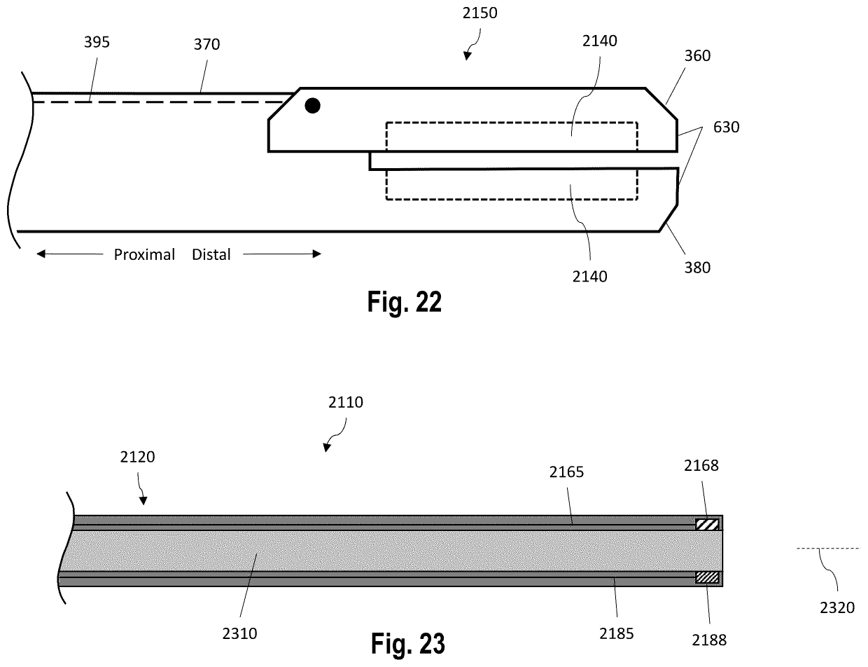

FIG. 22 is a schematic, diagrammatic side view of at least a portion of an example intraluminal tissue engagement sensor device 2150, according to aspects of the present disclosure. Visible are the first jaw 360, second jaw 380, flexible elongate member 370, and guide tracks or channel portions 2140. The flexible elongate rod 2120, emitter 2168, and receiver 2188 are configured to travel longitudinally through the guide tracks or channel portions 2140.

FIG. 23 is a schematic, diagrammatic, side cross-sectional view of an example movable tissue engagement sensor assembly 2110, according to aspects of the present disclosure. The movable tissue engagement sensor assembly 2110 includes a flexible elongate pusher/puller rod 2120, emitter 2168, emitter control wire(s) 2165, receiver 2188, receiver control wire(s) 2185, and a gap, split, or lumen 2310 in the flexible elongate rod 2120. The emitter 2168 and receiver 2188 are fixedly attached to the rod 2120. The movable tissue engagement sensor assembly 2110 is configured to slide longitudinally (e.g., along longitudinal axis 2320) through the guide tracks or channel portions 2140 of FIG. 22, such that the emitter 2168 and receiver 2188 are moved longitudinally along the jaws 360, 380.

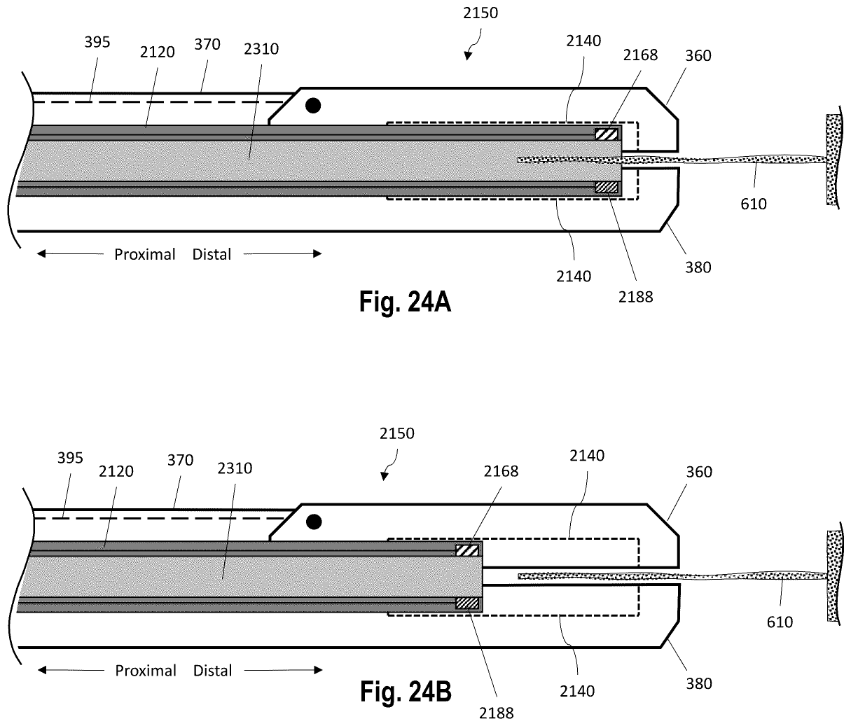

FIG. 24A is a diagrammatic, side cross-sectional view of an example intraluminal tissue engagement sensor device 2150 measuring tissue 610 (e.g., a heart valve leaflet), according to aspects of the present disclosure. The tissue 610 is grasped between the jaws 360, 380 and received into the gap or lumen 2310 in the flexible elongate rod 2120, such that the emitter 2168 and receiver 2188 are at a first location (e.g., the most distal end of their possible travel within the guide tracks or channel portions 2140). The emitter 2168 and receiver 2188 can be used to measure the presence, thickness, and/or characteristics of the tissue 610, as described above.

FIG. 24B is a diagrammatic, side cross-sectional view of an example intraluminal tissue engagement sensor device 2150 measuring tissue 610 (e.g., a heart valve leaflet), according to aspects of the present disclosure. The tissue 610 is grasped between the jaws 360, 380 and the flexible elongate rod 2120 is pulled back until, at a second location, the emitter 2168 and receiver 2188 no longer detect the presence of the tissue 610. The position of the flexible elongate rod 2120 (as measured by the linear encoder 2130 of FIG. 21) with respect to the distal end of its travel can then be used to deduce the length of tissue engaged by the device. In some cases, the flexible elongate rod 2120 may be pushed forward rather than pulled back in order to measure the length of the tissue 619 gripped by the jaws 360, 380.

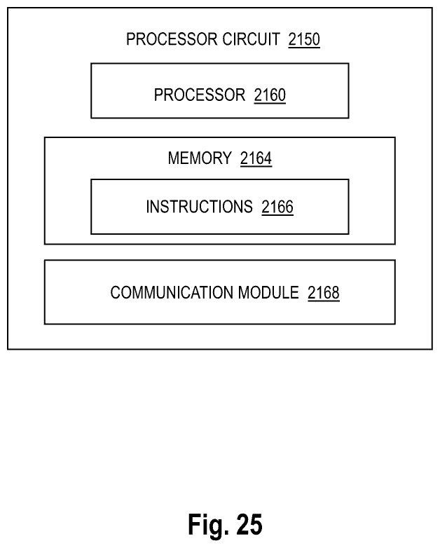

FIG. 25 is a schematic diagram of a processor circuit 2550, according to aspects of the present disclosure. The processor circuit 2550 may be implemented in processor circuit 310, the system 300, the system 1100, the system 1400, the system 1900, the system 2100, or other devices or workstations (e.g., third-party workstations, network routers, etc.), or on a cloud processor or other remote processing unit, as necessary to implement the method. As shown, the processor circuit 2550 may include a processor 2560, a memory 2564, and a communication module 2568. These elements may be in direct or indirect communication with each other, for example via one or more buses.

The processor 2560 may include a central processing unit (CPU), a digital signal processor (DSP), an ASIC, a controller, or any combination of general-purpose computing devices, reduced instruction set computing (RISC) devices, application-specific integrated circuits (ASICs), field programmable gate arrays (FPGAs), or other related logic devices, including mechanical and quantum computers. The processor 2560 may also comprise another hardware device, a firmware device, or any combination thereof configured to perform the operations described herein. The processor 2560 may also be implemented as a combination of computing devices, e.g., a combination of a DSP and a microprocessor, a plurality of microprocessors, one or more microprocessors in conjunction with a DSP core, or any other such configuration.

The memory 2564 may include a cache memory (e.g., a cache memory of the processor 2560), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 2564 includes a non-transitory computer-readable medium. The memory 2564 may store instructions 2566. The instructions 2566 may include instructions that, when executed by the processor 2560, cause the processor 2560 to perform the operations described herein. Instructions 2566 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements.

The communication module 2568 can include any electronic circuitry and/or logic circuitry to facilitate direct or indirect communication of data between the processor circuit 2550, and other processors or devices. In that regard, the communication module 2568 can be an input/output (I/O) device. In some instances, the communication module 2568 facilitates direct or indirect communication between various elements of the processor circuit 2550 and/or the system 300, 1100, 1400, 1900, or 2100. The communication module 2568 may communicate within the processor circuit 2550 through numerous methods or protocols. Serial communication protocols may include but are not limited to United States Serial Protocol Interface (US SPI), Inter-Integrated Circuit (I2C), Recommended Standard 232 (RS-232), RS-485, Controller Area Network (CAN), Ethernet, Aeronautical Radio, Incorporated 429 (ARINC 429), MODBUS, Military Standard 1553 (MIL-STD-1553), or any other suitable method or protocol. Parallel protocols include but are not limited to Industry Standard Architecture (ISA), Advanced Technology Attachment (ATA), Small Computer System Interface (SCSI), Peripheral Component Interconnect (PCI), Institute of Electrical and Electronics Engineers 488 (IEEE-488), IEEE-1284, and other suitable protocols. Where appropriate, serial and parallel communications may be bridged by a Universal Asynchronous Receiver Transmitter (UART), Universal Synchronous Receiver Transmitter (USART), or other appropriate subsystem.

External communication (including but not limited to software updates, firmware updates, preset sharing between the processor and central server, or readings from the tissue gripping device with tissue engagement sensor) may be accomplished using any suitable wireless or wired communication technology, such as a cable interface such as a universal serial bus (USB), micro USB, Lightning, or Fire Wire interface, Bluetooth, Wi-Fi, ZigBee, Li-Fi, or cellular data connections such as 2G/GSM (global system for mobiles), 3G/UMTS (universal mobile telecommunications system), 4G, long term evolution (LTE), WiMax, or 5G. For example, a Bluetooth Low Energy (BLE) radio can be used to establish connectivity with a cloud service, for transmission of data, and for receipt of software patches. The controller may be configured to communicate with a remote server, or a local device such as a laptop, tablet, or handheld device, or may include a display capable of showing status variables and other information. Information may also be transferred on physical media such as a USB flash drive or memory stick.

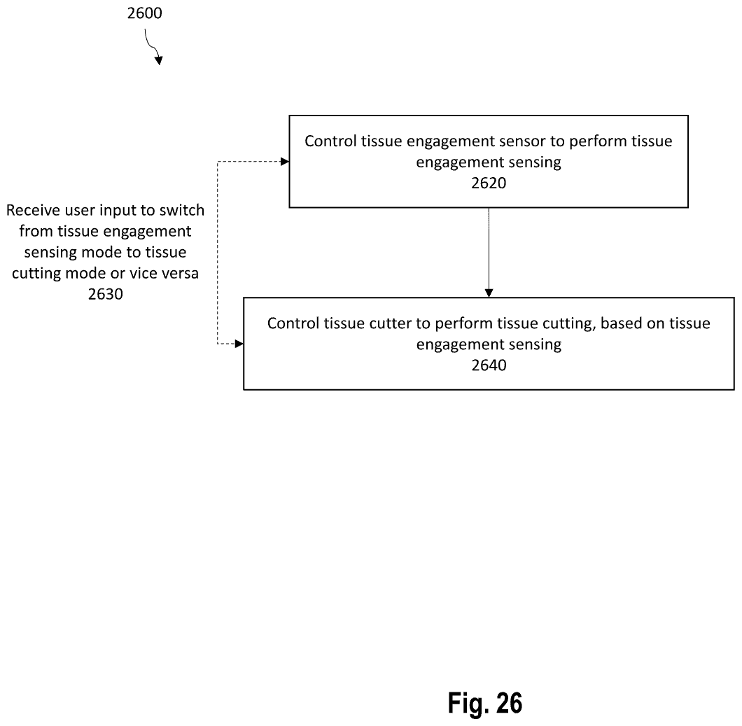

FIG. 26 is a schematic, diagrammatic representation, in flow diagram form, of an example tissue engagement and tissue cutting method 2600, according to aspects of the present disclosure.

In step 2620, the method 2600 includes controlling the tissue engagement sensor to perform tissue engagement sensing. Execution then proceeds to step 2630. Example tissue engagement sensors are described herein (e.g., in FIGS. 3-4B, 6-7, 12-21, 23-24B, above).

In step 2630, the method 2600 includes receiving a user input to switch from the tissue engagement sensing mode to a tissue cutting mode or vice-versa. Execution then proceeds to step 2620 or step 2640, depending on the input.

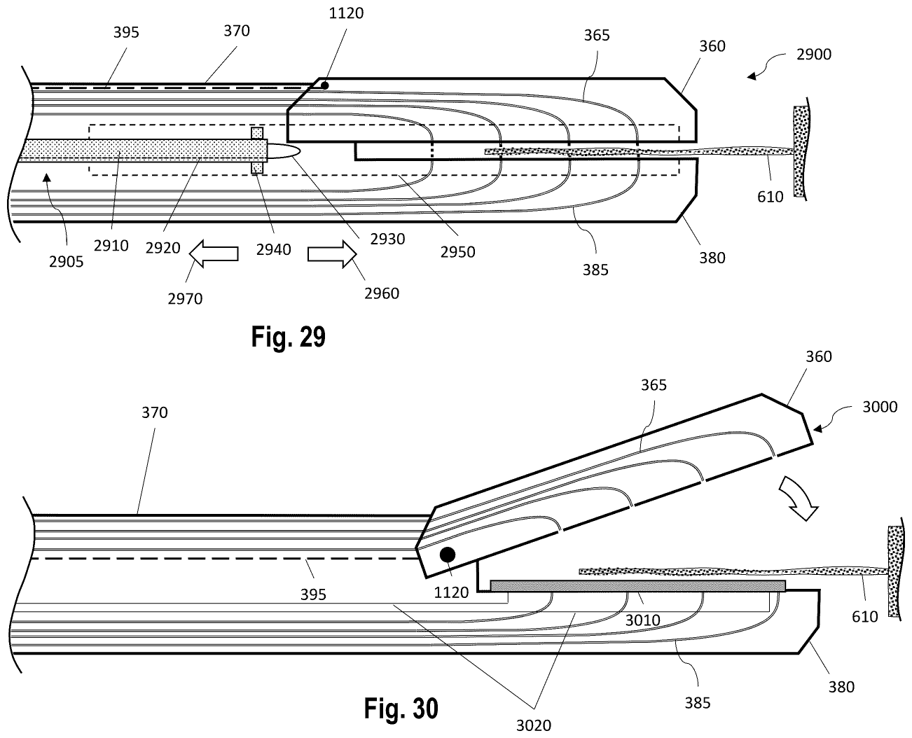

In step 2640, the method 2600 includes controlling a tissue cutter to perform tissue cutting, based on the tissue engagement sensing. The method then includes waiting for a user input. Execution then proceeds to step 1730. Tissue cutting can be referred to as tissue slitting, tissue resection, etc. Examples of the tissue cutter are described in U.S. Provisional Application No. 63/523,715, filed Jun. 28, 2023, entitled “HEART VALVE LEAFLET GRABBING AND SLITTING DEVICE”, and in U.S. Provisional Application No. 63/527,871, filed Jul. 20, 2023, entitled “MULTIPLE ELECTRODE HEART VALVE SLITTING DEVICE”, each of which is incorporated by reference as though fully set forth herein. Examples of controlling a tissue cutter based on tissue engagement sensing are described for example in FIGS. 29 and 30, below.

It is noted that the tissue engagement sensor and/or tissue cutter can be different components (whether parts of different devices or parts of the same device (as shown for example in FIGS. 29 and 30, below.

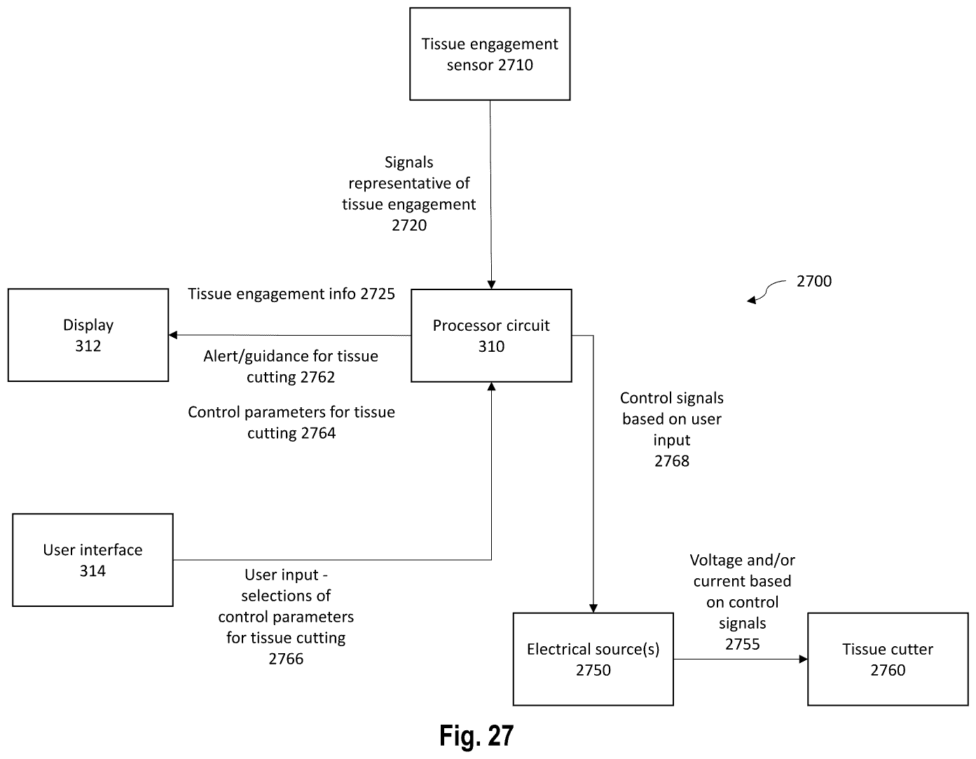

FIG. 27 is a schematic, diagrammatic representation, in block diagram form, of an example tissue engagement and tissue cutting system 2700, according to aspects of the present disclosure. In the example shown in FIG. 27, the system 2700 includes a tissue engagement sensor 2710 that generates signals 2720 that are representative of the sensed tissue engagement, and which are received by a processor circuit 310.

Based on the signals 2720, the processor circuit generates tissue engagement information 2725, alerts or guidance 2762 for tissue cutting, and/or control parameters 2764 for tissue cutting, any of which may be sent to the display 312. Via a user interface 314, the processor circuit 310 also receives user inputs 2766 regarding a selection of control parameters for tissue cutting. The processor circuit 310 then sends the selected control signals 2768 to an electrical source 2750 (or multiple electrical sources, depending on the implementation). The electrical source 2750 then sends a voltage and/or current 2755 based on the control signals to a tissue cutter 2760, which performs the tissue cutting.

It is noted that the user interface 314 and the display 312 can be the same component (e.g., a touch screen display), or can be separate components (e.g., a display plus knobs, switches, keyboard, mouse, etc.).

The processor circuit 310 generates the alert/guidance 2762 for tissue cutting based on the tissue engagement information 2725—e.g., recommendations for control parameters for electrical energy for cutting (amplitude, frequency, voltage/current, pulse length, pulse width, duty cycle, etc.). In some aspects, the alert/guidance 2762 for tissue cutting can be the tissue engagement information 2725, and may for example include text, symbols, or sound, and can be binary (e.g., yes/no, ready for cutting/not ready for cutting). The alert/guidance 2762 for tissue cutting can include text/symbol/sound indicating how much tissue engagement there is (e.g., where tissue engagement is, e.g., along the length of the sensor), whether a threshold amount of tissue engagement has been met, etc. For example, if the threshold is 75% engagement of the length along electrodes, then an engagement of greater than 75% may result in an output text/symbol/sound indicating the system is ready for cutting, whereas an engagement of less than 75% may result in an output text/symbol/sound indicating the system is not ready for cutting, and/or that the user should move the flexible elongate member with the tissue engagement sensor 2710 and/or tissue cutter 2760 to get more tissue engagement.

The system 2700 may include a screen display showing options for control parameters for electrical energy in tissue cutting (e.g., amplitude, frequency, voltage/current, pulse length, pulse width, duty cycle, etc.) Part of the guidance 2762 for tissue cutting can be providing automatically selected recommended control parameters (e.g., based on the tissue engagement at different locations), and then asking user for confirmation or changes. With the user interface 314, the user can provide user input to either confirm the auto-selected recommended control parameters, or make changes.

Via the user interface 314, the user provides user input 2766 representative of selections of the control parameters for tissue cutting. This can be based on the tissue engagement information 2725, the alert/guidance 2762 for tissue cutting, auto-selection of recommended control parameters, etc.

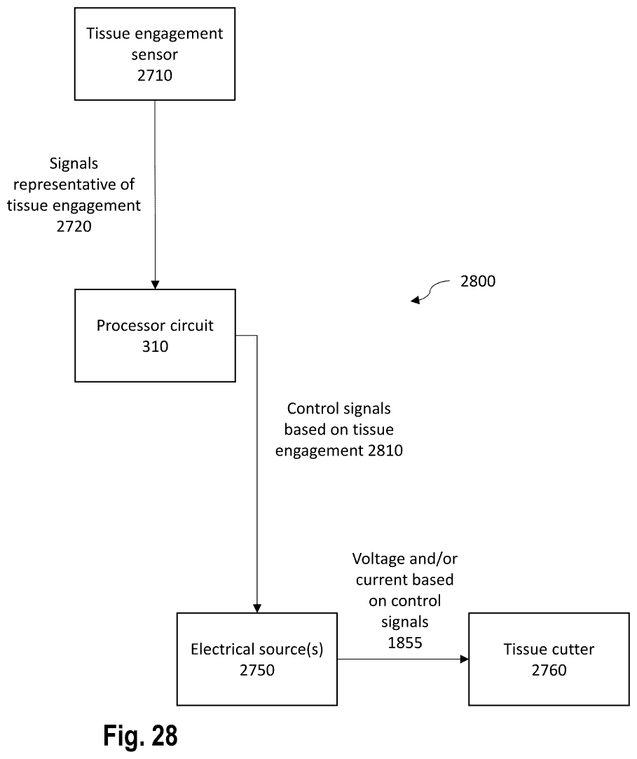

FIG. 28 is a schematic, diagrammatic representation, in block diagram form, of an example tissue engagement and tissue cutting system 2800, according to aspects of the present disclosure. FIG. 28 is similar to FIG. 27, except that the display and user interface have been eliminated, and the control signals 2768 based on user inputs have been replaced with control signals 2810 automatically selected by the processor circuit 310 based on the signals 2720 representative of tissue engagement.

In the example shown in FIG. 28, the processor circuit 310 controls activation of the tissue cutter based on tissue engagement information that is computed but not necessarily communicated to the user. The information may for example include off/on, ready/not ready information, as described above in FIG. 27. The processor circuit then determines control parameters for the electrical energy used for cutting (e.g., amplitude, frequency, voltage/current, pulse length, pulse width, duty cycle, etc.). If ready, then processor circuit 310 provides the control signals 2810 to the electrical source 2750 based on the determined control parameters, to activate the electrical source 2750 (e.g., cause the electrical source 2750 to output electrical energy to the tissue cutter 2760 for cutting).

If the system is not ready (e.g., less than 75% tissue engagement, etc.), then the processor circuit 310 prevents the electrical source 2750 from being activated (e.g., if the user tries to activate cutting, e.g., to output electrical energy to tissue cutter 2760 for cutting), or else the processor circuit 310 requires user confirmation to proceed (e.g., via a user override) with the cutting even if the system is not ready (e.g., less than 75% tissue engagement, etc.). When activated, the electrical source 2750 provides voltage and/or current based on the control signals 2810, which result in control parameters (amplitude, frequency, voltage/current, pulse length, pulse width, duty cycle, etc.) that are appropriate for cutting the engaged tissue.

FIG. 29 is a schematic, diagrammatic side cross-sectional view of an example tissue grasping, tissue engagement sensing and tissue cutting device 2900, according to aspects of the present disclosure. Visible are the upper jaw 360, lower jaw 380, hinge 1120, flexible elongate member 370, pull wire 395, and fiber optic cables 365, 385.

The tissue engagement sensing and tissue cutting device 2900 is similar in some regards to the tissue grasping and slitting device described in U.S. Provisional Application No. 63/523,715, filed Jun. 28, 2023, entitled “HEART VALVE LEAFLET GRABBING AND SLITTING DEVICE”, which is incorporated by reference as though fully set forth herein. The tissue engagement sensing and tissue cutting device 2900 is also similar in some regards to the tissue grasping and tissue engagement sensing device of FIGS. 4A-7, except that a cutting wire assembly 2905 has been added.

The cutting wire assembly 2905 includes a body 2910 (which may for example be a catheter), a conductor 2920 coupled to a cutting wire loop 2930, and guide pegs 2940 configured to slide within guide track(s) or channel(s) 2950 in both a distal direction 2960 and a proximal direction 2970. The conductor 2920 carries electrical energy (voltage and/or current, e.g., alternating at radio frequencies (RF)), thereby energizing and heating the cutting wire loop 2930 such that it can slit tissue (e.g., the tissue of a heart valve leaflet). In some aspects, wire loop 2930 can be an exposed portion of conductor 2920 (e.g., wire loop 2930 and conductor 2920 may be different portions of the same component). In other aspects, wire loop 2930 can be coupled to conductor 2920 (e.g., wire 2930 may be a separate component that is mechanically and electrically coupled to the conductor 2920).

In the example shown in FIG. 29, the cutting wire assembly 2905 is in a retracted position, such that the cutting wire loop 2930 does not extend outside the flexible elongate member 370. When the cutting wire assembly 2905 is in an extended position, the cutting wire loop 2930 extends outside the flexible elongate member 370, along the guide track 2950 between the upper jaw 360 and lower jaw 380. This extended configuration allows slitting of the tissue 610 that is gripped between the upper jaw 360 and lower jaw 380.

It is noted that the cutting assembly 2905 shown in FIG. 29 is a monopolar cutting design, where the current passes from the generator, through the device, into tissue in contact with the device, through the body, and into a return electrode. However, bi-polar designs, wherein at least one of the two jaws provides a return path or ground path, also fall within the scope of the present disclosure.

FIG. 30 is a schematic, diagrammatic side cross-sectional view of an example tissue grasping, tissue engagement sensing, and tissue cutting device 3000, according to aspects of the present disclosure. Visible are the upper jaw 360, lower jaw 380, fiber optic cables 365, fiber optic cables 385, hinge 1120, flexible elongate member 370, and pull wire 395.

The tissue engagement sensing and tissue cutting device 3000 is similar in some regards to the tissue grasping and slitting device described in U.S. Provisional Application No. 63/523,715, filed Jun. 28, 2023, entitled “HEART VALVE LEAFLET GRABBING AND SLITTING DEVICE”, which is incorporated by reference as though fully set forth herein. The tissue engagement and tissue cutting device 3000 is similar in some regards to the tissue grasping and tissue engagement sensing device of FIGS. 4A-7, except that a cutting electrode 3010, controlled by wires 3020, has been added. When energized with radio frequency (RF) electrical energy by the wires 3020, the cutting electrode 3010 is capable of cutting the tissue 610 that is grasped between the upper jaw 360 and lower jaw 380, when the jaws are closed.

It is noted that although FIGS. 29 and 30 show the tissue engagement sensor including the optical fibers from FIG. 4A-7, it could, instead or in addition, include the LED emitters and receivers of FIGS. 12-13 and/or the ultrasound emitters and receivers of FIG. 20, or other related sensor types, without departing from the spirit of the present disclosure.

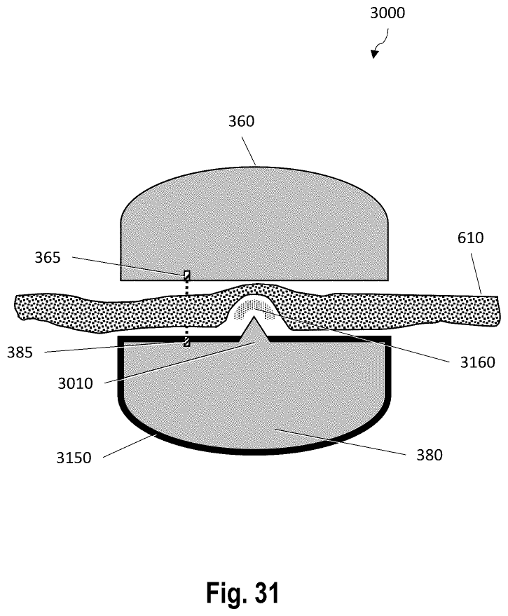

FIG. 31 is a schematic, diagrammatic end cross-sectional view of the example tissue grasping, tissue engagement sensing, and tissue cutting device 3000 of FIG. 30, according to aspects of the present disclosure. Visible are the upper jaw 360, lower jaw 380, fiber optic cables 365, fiber optic cables 385, cutting electrode 3010, and tissue 610. Also visible are an insulative coating 3150, and a cutting plasma 3160 that is generated by the cutting electrode 3010 and that cuts the tissue 610.

As will be readily appreciated by those having ordinary skill in the art after becoming familiar with the teachings herein, the tissue gripping device with tissue engagement sensor advantageously permits a clinician (e.g., a heart surgeon) to grasp and immobilize a heart valve leaflet or other body tissue, with confidence about the amount of tissue grasped, and thus minimal risk of damage to the heart wall or other adjacent tissues. The technology may also be used for example to measure the size of heart valve leaflets or other tissue. In some aspects, the emitters and/or sensors may be disposed within a lumen (e.g., the central lumen of a catheter). In some aspects, the emitters may be located on a first guidewire and the receivers may be located on a second guidewire. In some aspects, the emitters and/or sensors may be ring-shaped rather than pad-shaped. Any type of emitters and any type of complementary sensors may be used without departing from the spirit of the present disclosure.

Although intended for specific treatment of heart valve related diseases, the technology disclosed herein could be expanded to anywhere tissue needs to be remotely grasped, such as endoscopic, laparoscopic, intravascular, intraluminal, and/or robotic surgical procedures. Although intended for intravascular use, especially for transcatheter edge-to-edge repair (TEER) type applications, the systems, devices, and methods disclosed herein are also applicable to any instance where the amount of tissue grasped between a set of jaws is desirable information, such as endoscopic surgery, or any other procedure where direct visualization is not feasible.

The logical operations making up the aspects of the technology described herein are referred to variously as operations, steps, objects, elements, components, or modules. Furthermore, it should be understood that these may be arranged or performed in any order, unless explicitly claimed otherwise or a specific order is inherently necessitated by the claim language. It should further be understood that the described technology may be employed in single-use and multi-use devices for medical or nonmedical use.

All directional references e.g., upper, lower, inner, outer, upward, downward, left, right, lateral, front, back, top, bottom, above, below, vertical, horizontal, clockwise, counterclockwise, proximal, and distal are only used for identification purposes to aid the reader's understanding of the claimed subject matter, and do not create limitations, particularly as to the position, orientation, or use of aspects of the present disclosure. Connection references, e.g., attached, coupled, connected, and joined are to be construed broadly and may include intermediate members between a collection of elements and relative movement between elements unless otherwise indicated. As such, connection references do not necessarily imply that two elements are directly connected and in fixed relation to each other. The term “or” shall be interpreted to mean “and/or” rather than “exclusive or.” The word “comprising” does not exclude other elements or steps, and the indefinite article “a” or “an” does not exclude a plurality. Unless otherwise noted in the claims, stated values shall be interpreted as illustrative only and shall not be taken to be limiting.

The above specification, examples and data provide a complete description of the structure and use of exemplary aspects of the present disclosure, e.g., as defined in the claims. Although various aspects of the claimed subject matter have been described above with a certain degree of particularity, or with reference to one or more individual aspects, those skilled in the art could make numerous alterations to the disclosed aspects without departing from the spirit or scope of the claimed subject matter.

Still other aspects are contemplated. It is intended that all matter contained in the above description and shown in the accompanying drawings shall be interpreted as illustrative only of particular aspects and not limiting. Changes in detail or structure may be made without departing from the basic elements of the subject matter as defined in the following claims.

What is claimed is:

1. An apparatus, comprising:
an intracardiac tissue engagement sensor configured to sense engagement with a valve leaflet of a heart valve, wherein the intracardiac tissue engagement sensor comprises:
a first jaw and a second jaw configured for movement relative to one another, wherein the first jaw and the second jaw are configured to receive a valve leaflet in a space between the first jaw and the second jaw;
a first emitter coupled to the first jaw, wherein the first emitter is configured to emit first energy in an unmodified state; and
a first receiver coupled to the second jaw, wherein the first receiver is configured to receive the first energy in a modified state or not receive the first energy based on interaction with the valve leaflet that is positioned between the first emitter and the first receiver.

2. The apparatus of claim 1, wherein, when the first jaw and the second jaw are in a closed state, the first emitter and the first receiver are aligned along a length of the intracardiac tissue engagement sensor.

3. The apparatus of claim 1,
wherein the intracardiac tissue engagement sensor further comprises:
a second emitter disposed on the first jaw and spaced from the first emitter along a length of the first jaw; and
a second receiver disposed on the second jaw and spaced from the first emitter along a length of the second jaw,
wherein, when the first jaw and the second jaw are in the closed state, the second emitter and the second receiver are aligned along the length of the first jaw and the length of the second jaw.

4. The apparatus of claim 3,
wherein the second emitter is configured to emit second energy in the unmodified state,
wherein the second receiver is configured to receive the second energy in the unmodified state when the valve leaflet is not positioned between the second emitter and the second receiver.

5. The apparatus of claim 1,
further comprising a processor configured for communication with the intracardiac tissue engagement sensor,
wherein the processor is configured to determine an amount of the engagement between the valve leaflet and the intracardiac tissue engagement sensor, based on first receiver receiving the first energy in the modified state or not receiving the first energy.

6. The apparatus of claim 1, wherein the first energy comprises at least one of light or ultrasound.

7. The apparatus of claim 1, wherein the modified state comprises a change in intensity, wavelength, or polarization as compared with the unmodified state.

8. The apparatus of claim 1,
further comprising a catheter,
wherein an intracardiac tissue engagement sensor is permanently coupled to the catheter.

9. The apparatus of claim 1,
further comprising a catheter,
wherein an intracardiac tissue engagement sensor is implantable within a heart,
wherein the intracardiac tissue engagement sensor is removably coupled to the catheter.

10. The apparatus of claim 1, further comprising a tissue cutter configured to slit the heart valve leaflet.

11. The apparatus of claim 10, wherein the tissue cutter is controllable to cut the valve leaflet based on whether the valve leaflet is positioned between the first emitter and the first receiver.

12. A tissue gripping and measurement device, comprising:
a catheter configured to be positioned within a body of a patient;
a gripping assembly coupled to the catheter and comprising:
a first jaw;
a second jaw;
a hinge mechanism coupled to the first jaw and the second jaw and configured to rotate the first jaw relative to the second jaw between an open state and a closed state; and
a first emitter disposed on the first jaw or the second jaw; and
a first receiver disposed on the other of the first jaw or the second jaw,
wherein when tissue is not fully gripped by the gripping assembly, first energy emitted by the first emitter is received by the first receiver in an unmodified state, and
wherein when tissue is fully gripped by the gripping assembly, the first energy emitted by the first emitter is received by the first receiver in a modified state.

13. The tissue gripping and measurement device of claim 12, further comprising:
a second emitter disposed on the first jaw or the second jaw; and
a second receiver disposed on the other of the first jaw or the second jaw,
wherein when tissue is not gripped by the gripping assembly, second energy emitted by the second emitter is received by the second receiver in the unmodified state, and
wherein when tissue is partially gripped by the gripping assembly, the second energy emitted by the first emitter is received by the first receiver in the modified state.

14. The tissue gripping and measurement device of claim 13, wherein the second energy is emitted by the second emitter at a wavelength or polarization different from the wavelength or polarization of the first energy emitted by the first emitter.

15. The tissue gripping and measurement device of claim 12,
wherein the first emitter is an optical fiber coupled to a light source, and
wherein the first receiver is an optical fiber coupled to a light sensor.

16. The tissue gripping and measurement device of claim 12,
wherein the first emitter is an LED, and
wherein the first receiver is a photodiode.

17. The tissue gripping and measurement device of claim 12,
wherein the first emitter is an ultrasound emitter, and
wherein the first receiver is an ultrasound receiver.

18. The tissue gripping and measurement device of claim 12, wherein the modified state comprises a change in intensity, wavelength, or polarization as compared with the unmodified state.

19. The tissue gripping and measurement device of claim 12, wherein the tissue is a heart valve leaflet, and wherein the gripping assembly is configured to grip the heart valve leaflet.

20. The tissue gripping and measurement device of claim 19, wherein the gripping assembly, the first emitter, and the first receiver are configured to remain within the body after gripping the heart valve leaflet.

21. The tissue gripping and measurement device of claim 12, wherein the hinge mechanism comprises a hinge pin and a pull wire.

22. The tissue gripping and measurement device of claim 12, wherein the processor is configured to detect whether the tissue is fully gripped by the gripping assembly based on whether the first energy received by the first receiver is received in the modified state or the unmodified state.

23. The tissue gripping and measurement device of claim 12, further comprising a tissue cutter configured to cut the tissue.

24. The tissue gripping and measurement device of claim 23, wherein the tissue cutter is controllable to cut the tissue based on whether the first energy emitted by the first emitter is received by the first receiver in a modified state.
